(19) United States
(12) Patent Application Publication
(10) Pub. No.: US 2025/0122479 A1
(43) Pub. Date: Apr. 17, 2025

(54) GENERATION OF LUNG ORGANOIDS FROM PLURIPOTENT STEM CELLS AND USE THEREOF

(71) Applicant: THE TRUSTEES OF COLUMBIA UNIVERSITY IN THE CITY OF NEW YORK, New York, NY (US)

(72) Inventors: Hans-Willem SNOECK, New York, NY (US); Ivana MATKOVIC LEKO, New York, NY (US); Mikael PEZET, New York, NY (US)

(73) Assignee: THE TRUSTEES OF COLUMBIA UNIVERSITY IN THE CITY OF NEW YORK, New York, NY (US)

(21) Appl. No.: 18/967,738

(22) Filed: Dec. 4, 2024

Related U.S. Application Data
(63) Continuation of application No. PCT/US2023/067960, filed on Jun. 6, 2023.
(60) Provisional application No. 63/349,205, filed on Jun. 6, 2022.

(51) Int. Cl. C12N 5/071; A61K 35/42; C12N 5/00; G01N 33/50

(52) U.S. Cl. CPC C12N 5/0689; A61K 35/42; C12N 5/0018; G01N 33/5044; C12N 2501/01; C12N 2501/117; C12N 2501/119; C12N 2501/155; C12N 2501/385; C12N 2501/39; C12N 2501/727; C12N 2501/73; C12N 2503/02; C12N 2506/03; C12N 2513/00; G01N 2500/10

(57) ABSTRACT

Disclosed herein are lung progenitor cells that are in the form of lung organoids and methods of generating the lung progenitor cells. The lung organoids can be an important resource for treating lung disorders and injuries, studies in human lung regeneration, disease modeling, and drug target identification and validation.

### CROSS-REFERENCE TO RELATED APPLICATIONS

The present application is a Continuation of International application PCT/US2023/067960, filed on Jun. 6, 2023, which claims priority to U.S. Provisional Patent Application No. 63/349,205 filed on Jun. 6, 2022, which is incorporated herein by reference in its entirety.

### STATEMENT OF GOVERNMENT SUPPORT

This invention was made with government support under U01HL134760 awarded by the National Institutes of Health. The government has certain rights in the invention.

### FIELD OF THE INVENTION

The present invention relates to methods of generating lung progenitor cells (e.g., transitional lung organoids). The lung progenitor cells may be used to model lung diseases/conditions and screen for desired agents. The lung progenitors can be used as therapeutic treatments for various pulmonary disorders or injuries.

### BACKGROUND

Several novel distal lung populations were recently identified in humans that may be involved in regeneration after injury.¹⁻³ Although the lung displays extensive regenerative capacity, the mechanism underlying the extensive regenerative capacity of the lung are unclear. Furthermore, diseases such as idiopathic pulmonary fibrosis (IPF), are characterized by aberrant repair after injury, genetic or environmental, to alveolar type 2 (AT2) cells.

The respiratory epithelium contains basal (BC), ciliated, secretory, goblet and neuroendocrine cells in the airways, and alveolar type 1 (AT1) and surfactant-producing AT2 cells in the alveoli, where gas exchange takes place.^{4,5} Mouse studies have shown multiple layers of often facultative progenitors involved in regeneration.⁶ AT2 cells,⁷ and most likely a subset thereof,^{8,9} can differentiate into type 1 cells, although rare AT1 cells can participate in alveolar regrowth after partial pneumonectomy.¹⁰ After severe injury, a variety of distal airway cells that share features with BCs or with secretory cells participate in regeneration,¹¹⁻¹⁹ although other studies did not show contribution of cells traced with the secretory marker, Scgb1a1, to alveolar repair.²⁰

In humans, single cell RNA sequencing (scRNASeq) studies have suggested that AT2-to-AT1 plasticity also exists.^{1-3,21} In lungs of patients with IPF, aberrant basaloid cells (KRT17⁺KRT5⁻, and expressing mesenchymal markers) are observed,^{1,22} likely a consequence of abnormal repair. In contrast to mice, primates and ferrets have “terminal respiratory bronchioles” or “respiratory airways” into which multiple alveoli coalesce, and where transitional populations were identified that may play a role in regeneration, as they were more abundant after lung injury and in fibrotic lungs.¹⁻³ A feature of at least some of these populations is the expression of the secretory cell marker, SCGB3A2, first reported by Haberman et al. who found SCGB3A2⁺ cells co-expressing AT1 and AT2 markers in the distal lung.¹ Basil et al. also reported SCGB3A2⁺ cells negative for the AT2 marker, SFPTC, in respiratory airways.² Murthy et al. identified multiple subpopulations characterized by co-expression of another AT2 marker, SFTPB.³ These included SCGB3A2⁺SFTPB⁺SFTPC⁺“AT0” cells, SFTPB⁺SCGB3A2⁺SFTPC⁻ terminal respiratory bronchiole secretory cells (TRB-SCs), SFTPB⁺SCGB3A2⁺SCGB1A1⁺ pre-TRB-SCs, and SFPTB⁺SCGB3A2⁻ distal BCs, some of which are low to negative for the airway BC markers, KRT5 and p63.

The lineage relations between these cell types are unclear. Based on airway organoids derived from human pluripotent stem cells (hPSCs), Basil et al.² proposed unidirectional differentiation of SCGB3A2⁺ cells into AT2 cells, which was consistent with their Slingshot²³ pseudotime analysis. On the other hand, Murthy et al.,³ using adult distal lung organoids, suggested that AT0 cells are transitional between AT2 and AT1 fates and can also give rise to TRB-SCs, a conclusion supporting their own Velocity trajectory analysis but inconsistent with findings of Basil et al.²⁴, and raising the question to what extent the SCGB3A2⁺ cells in the organoids truly corresponded to populations expressing the same marker in the distal lung.

Currently available organoid models yielded conflicting data and these cell types are not present in mice^{2,3}. Platforms that include three-dimensional (3D) tissue constructs, or organoids, created using mammalian (e.g., human) cells offer a better solution for mimicking native physiology, modeling diseases, and performing drug screening. Therefore, there is a need for an in vitro model that contains these distal progenitor cell populations.

Regenerative medicine holds promise for new treatment options. Diseases that are amenable to cellular therapies encompass both airway and distal lung disease. Among distal lung diseases, many affect the function of type 2 alveolar epithelial cells. Replacing those with stem cell-derived, patient-specific and genetically corrected cells may provide improvement or even cure. One such distal lung disease is idiopathic pulmonary fibrosis (IPF). The notion that defects in AT2 cells underlie IPF is further supported by the fact that patients with Hermansky-Pudlak Syndrome (HPS) show a high incidence of IPF, also called HPS-associated interstitial pneumonia (HPSIP). Novel approaches for cell replacement therapy for lung diseases are urgently needed.

### SUMMARY

The present disclosure provides for a method for generating lung progenitor cells. The method may comprise: (a) producing anterior foregut endoderm cells from mammalian pluripotent stem cells (PSCs); (b) culturing the anterior foregut endoderm cells in a suspension culture comprising a glycogen synthase kinase (GSK) inhibitor, a bone morphogenic protein (BMP) agonist, one or more FGF agonists, and retinoic acid (or its derivative), to generate at least one lung bud organoid (LBO); (c) culturing the LBO in a three-dimensional (3D) matrix in the presence of a GSK inhibitor, a BMP agonist, one or more FGF agonists, and retinoic acid (or its derivative), to form a branched LBO (BLBO); and (d) dissociating the LBO or BLBO, and culturing the dissociated LBO or BLBO in a 3D matrix in the presence of a GSK3 inhibitor, an FGF agonist, a corticosteroid, a 3′,5′-cyclic adenosine monophosphate (cAMP) pathway activator, and a phosphodiesterase (PDE) inhibitor.

The GSK inhibitor may be CHIR99021. The GSK inhibitor (e.g., in step (b), step (c), and/or step (d)) may be at a concentration ranging from about 1 μM to about 10 μM, or about 3 μM.

The one or more FGF agonists may be FGF10 and keratinocyte growth factor (KGF).

The FGF agonist may be KGF.

The FGF agonist, or the one or more FGF agonists, may be at a concentration ranging from about 5 ng/ml to about 20 ng/ml, or about 10 ng/ml.

The corticosteroid may be dexamethasone. The corticosteroid may be at a concentration ranging from about 100 nM to about 150 nM, or about 127 nM.

The cAMP pathway activator may be cAMP or 8-bromo-cAMP. The cAMP pathway activator may be at a concentration ranging from about 0.05 mM to about 0.2 mM, or about 0.1 mM.

The PDE inhibitor may be 3-isobutyl-1-methylxanthine. The PDE inhibitor may be at a concentration ranging from about 0.05 mM to about 0.2 mM, or about 0.1 mM.

The BMP agonist may be BMP4.

In certain embodiments, the BMP agonist is BMP4, the one or more FGF agonists are KGF and FGF10. In certain embodiments, KGF, FGF10, and/or BMP4 are at a concentration of about 10 ng/ml.

Retinoic acid or its derivative may be at a concentration ranging from about 10 nM to about 100 nM, or about 50 nM.

The LBO or BLBO may be dissociated to single cells (e.g., in step (d)).

In certain embodiments, the method may further comprise generating a single cell suspension (e.g., in step (d)) before culturing the dissociated LBO or BLBO in the 3D matrix.

The present disclosure also provides for lung progenitor cells generated in vitro. The lung progenitor cells may be generated by the present method.

The lung progenitor cells may be in the form of lung organoids.

The lung progenitor cells may comprise type 0 alveolar epithelial (AT0) cells, terminal respiratory bronchiole stem cells (TRB-SCs) and distal basal cells (BCs). The lung progenitor cells may further comprise neuroendocrine cells. The lung progenitor cells may comprise (or consist essentially of, or consist of) type 0 alveolar epithelial (AT0) cells, terminal respiratory bronchiole stem cells (TRB-SCs), distal basal cells (BCs) and neuroendocrine cells.

The lung progenitor cells may comprise (or consist essentially of, or consist of) SCGB3A2⁺SFTPB⁺SFTPC⁺ cells, SFTPB⁺SCGB3A2⁺SFTPC⁻ cells, and SFPTB⁺SCGB3A2⁻ cells.

The present disclosure provides for an artificial lung organoid generated in vitro. The artificial lung organoid may be generated by the present method.

The artificial lung organoid may comprise type 0 alveolar epithelial (AT0) cells, terminal respiratory bronchiole stem cells (TRB-SCs) and distal basal cells (BCs). The artificial lung organoid may further comprise neuroendocrine cells. The artificial lung organoid may comprise (or consist essentially of, or consist of) type 0 alveolar epithelial (AT0) cells, terminal respiratory bronchiole stem cells (TRB-SCs), distal basal cells (BCs) and neuroendocrine cells.

The artificial lung organoid may comprise (or consist essentially of, or consist of) SCGB3A2⁺SFTPB⁺SFTPC⁺ cells, SFTPB⁺SCGB3A2⁺SFTPC⁻ cells, and SFPTB⁺SCGB3A2⁻ cells.

The lung progenitor cells or artificial lung organoid may express SFTPC and SFTPB. The lung progenitor cells or artificial lung organoid may express EPCAM, NKX2.1 and SFTPB. The lung progenitor cells or artificial lung organoid may express SCGB3A2. The lung progenitor cells or artificial lung organoid may express mRNAs encoding SCGB3A2, SFTPC, SFTPB, ABCA3, LPCAT, NAPSA, SLC234A2, LAMP3, or combinations thereof.

Also encompassed by the present disclosure is a cell population comprising the lung progenitor cells, or the artificial lung organoid.

The present disclosure also provides for a pharmaceutical composition comprising the lung progenitor cells, the cell population, or the artificial lung organoid.

The LBO may comprise (or consist essentially of, or consist of) (i) lung epithelial cells expressing FOXA2, FOXA1, NKX2.1 and EPCAM, and (ii) mesenchymal progenitors expressing PDGFRa, CD90, TBX4 and HOXA5.

The 3D matrix may be a solubilized basement membrane preparation from the Engelbreth-Holm-Swarm (EHS) mouse sarcoma. The 3D matrix may be Matrigel.

The mammalian PSCs may be human pluripotent stem cells (hPSCs). The mammalian PSCs may be embryonic stem cells (ESCs) and/or induced pluripotent stem cells (iPSCs).

The artificial lung organoid, the lung progenitor cells, or the cell population may differentiate into alveolar (like) cells after a withdrawal of CHIR99021 and KGF and in the presence of SB431542. The artificial lung organoid, the lung progenitor cells, or the cell population may differentiate into airway basal (like) cells when cultured on feeder cells in the presence of an inhibitor of Rho kinase (ROCK) and EGF.

The present disclosure provides for a method of treating a pulmonary disorder or injury in a subject in need thereof. The method may comprise administering to the subject an effective amount (e.g., a therapeutically effective amount) of the lung progenitor cells, the cell population, or the artificial lung organoid.

The pulmonary disorder or injury may be, cystic fibrosis; emphysema; chronic obstructive pulmonary disease (COPD); pulmonary fibrosis; idiopathic pulmonary fibrosis (IPF); Hermansky-Pudlak Syndrome; hypersensitivity pneumonitis; sarcoidosis; asbestosis; autoimmune-mediated interstitial lung disease; pulmonary hypertension; lung cancer; acute lung injury (adult respiratory distress syndrome); respiratory distress syndrome of prematurity, chronic lung disease of prematurity (bronchopulmonary dysplasia); surfactant protein B deficiency, surfactant protein C deficiency, ABCA3 deficiency; NKX2.1 mutation; ciliopathies; congenital diaphragmatic hernia; pulmonary alveolar proteinosis; pulmonary hypoplasia; lung injury, or combinations thereof.

The pulmonary disorder or injury may be an interstitial lung disease or a congenital surfactant deficiency.

The lung progenitor cells, the cell population, or the artificial lung organoid, may be non-syngeneic or syngeneic with the subject. The lung progenitor cells, the cell population, or the artificial lung organoid, may be allogeneic or xenogeneic with the subject. The lung progenitor cells, the cell population, or the artificial lung organoid, may be autologous with the subject.

Also encompassed by the present disclosure is a method of screening an agent for pharmacological or toxicological activity. The method may comprise: (a) administering an agent to the lung progenitor cells, the cell population, or the artificial lung organoid; and (b) assaying at least one pharmacological or toxicological response from at least one cell of the lung progenitor cells, the cell population, or the artificial lung organoid. The response may comprise fibrosis formation; cell death, cell growth, absorption of the agent, distribution of the agent, metabolism of the agent, excretion of the agent, and/or upregulation or downregulation of a substance by the at least one cell.

### BRIEF DESCRIPTION OF THE DRAWINGS

FIGS. 1a-1d. Generation of TLOs. FIG. 1a: Schematic representation of differentiation protocol (top) and representative bright field images of the various stages of the protocol (organoids: RUES2 ESCs, TLOs (right hand panels): iPSCs). FIG. 1b: Representative bright field images of TLOs generated from RUES2 ESCs. FIG. 1c: TLOs generated from mixed single cells from organoids expressing either GFP or mScarlet, showing that each TLO expresses a single reporter, indicating clonality. FIG. 1d: Expression of AT2 markers before and after cryopreservation of ESC-derived TLOs (n=3).

FIGS. 2a-2d. Characterization of TLOs. FIG. 2a: cRT-qPCR analysis of RUES2 ESC-derived and iPSC-derived TLOs (n=3) compared to lung bud organoids at day 21 (n=3, *P<0.05, students t-test). FIG. 2b: Representative immunofluorescence images of expression of indicated markers between 1 and 6 passages after generation from RUES2 ESCs and iPSCs. FIG. 2c, FIG. 2d: Representative Western Blot analysis of the expression of fully processed SFPTB and SFTPC in undifferentiated RUES2 cells, in two distinct lines of TLOs derived from RUES2 ESCs, and in human adult lung (FIG. 2c) and in an additional RUES2 ESC-derived TLO line as well as iPSC-derived TLO line (FIG. 2d).

FIGS. 3a-3d. Further characterization of TLOs. FIG. 3a: Transmission election microscopy of TLOs (at increasing magnification from left to right) of RUES2 ESC-derived TLOs. Stars: glycogen; arrows: LBs. Expression of Ki67 (FIG. 3b) and of cCASP3 (FIG. 3c) in TLOs in CK-DCI and 5 days after withdrawal of CHIR. FIG. 3d: RT-PCR of AT2 markers 1 to 5 days after withdrawal of CHIR (n=3).

FIGS. 4a-4g. scRNAseq analysis of TLOs. FIG. 4a: UMAP feature plots for indicated markers. FIG. 4b: Clustering analysis. FIG. 4c: Heatmap of top differentially expressed genes in each cluster identified in scRNAseq of ESC-derived TLOs. FIG. 4d: Cell identity assignment based on data of Murthy et al.³ FIG. 4e: scVelo latent time velocity analysis. FIG. 4f: Cell identity assignment based on data of Haberman et al.¹ FIG. 4g: Cell identity assignment based on data of Haberman et al.¹ with inclusion of data on IPF lungs.

FIGS. 5a-5k. Differentiation of TLOs. FIG. 5a: Schematic of protocol for distal differentiation. FIG. 5b: Representative images. FIG. 5c: RT-qPCR for AT2 (SFTPB, SFTPC, ABCA3) and AT1 (RAGE) markers. FIG. 5d: Flow cytometric quantification of AT1 and AT2 cells. FIG. 5e: Immunofluorescence (IF) of RAGE expression. FIG. 5f: Schematic of protocol for proximal differentiation. FIG. 5g: Representative images. FIG. 5h: Flow cytometric quantification of expression of BC markers (NGFR, CD104). FIG. 5i: Representative immunofluorescence images of expression of proximal markers. FIG. 5j: Quantification of FIG. 5i. FIG. 5k: RT-qPCR of expression of proximal (TP63) and distal (SFTPC) markers after proximal differentiation. (student's unpaired t-test).

FIGS. 6a-6d. Integrated scRNAseq analysis of WT, ABCA3^{-/-} and HPS1^{-/-} TLOs. FIG. 6a: Leiden plot showing differential presence of subpopulations (RUES2=WT parental line). FIG. 6b: Representative UMAP of differentially regulated genes. FIG. 6c: Select genes differentially regulated across genotypes. FIG. 6d: Pseudobulk unsupervised clustering and top differentially expressed pathways.

FIGS. 7a-7f. Functional phenotypes of WT, ABCA3^{-/-} and HPS1^{-/-} TLOs. FIG. 7a: Expression of AT1 (AGER) and AT2 (SFPTC, SFTPB, NAPSA) after induction of TLO differentiation into distal lineages by DCI+SB. FIG. 7b: Schematic of ER stress induction by tunicamycin. FIG. 7c: Expression of ER stress mediators after 5 hrs and 1 week after exposure to tunicamycin. FIG. 7d: Apoptosis 1 week after exposure to tunicamycin. FIG. 7e: Mitochondrial mass in WT and mutant TLOs in steady state. FIG. 7f: Schematic representation of ER stress and ISR pathways, and defects caused by HSP and ABCA3 deletion. R2=parental WT RUES ESC-derived cells.

### DETAILED DESCRIPTION

The present disclosure provides for lung progenitor cells that are in the form of lung organoids (e.g., transitional lung organoids or TLOs) and methods of generating the lung progenitor cells. The lung organoids can be used to create engineered 3D models that can more accurately recapitulate mammalian physiology and diseases. The lung organoids can be an important resource for studies in human lung regeneration, disease modeling, and drug target identification and validation. These lung progenitor cells may be used to engraft the lungs, serving as a regenerative therapy for treating various lung diseases, conditions, and injuries. The lung progenitor cells may also be used for identifying specific gene products or facets of disease states. The lung progenitor cells may be prepared from cells of subjects with mutation(s) and subsequently used to define relevant factor(s) associated with the mutation(s).

The present disclosure provides for a method for generating lung progenitor cells. The method may comprise: (a) producing anterior foregut endoderm cells from mammalian stem cells (e.g., mammalian pluripotent stem cells (PSCs)); (b) culturing the anterior foregut endoderm cells in a suspension culture to generate at least one lung bud organoid (LBO); (c) culturing the LBO in a three-dimensional (3D) matrix to form a branched LBO (BLBO), and (d) dissociating the LBO or BLBO and culturing the dissociated LBO or BLBO in a 3D matrix.

In certain embodiments, in step (d) of the method, the dissociated LBO or BLBO is cultured to generate the lung progenitor cells.

In certain embodiments, the present method for generating lung progenitor cells may comprise: (a) producing anterior foregut endoderm cells from mammalian stem cells (e.g., mammalian pluripotent stem cells (PSCs)); (b) culturing the anterior foregut endoderm cells in a suspension culture comprising a Wnt agonist (e.g., a glycogen synthase kinase (GSK) inhibitor such as CHIR99021, etc.), a bone morphogenic protein (BMP) agonist (e.g., BMP4), one or more FGF agonists (e.g., FGF10, keratinocyte growth factor (KGF)), and retinoic acid (or its derivative), to generate at least one lung bud organoid (LBO); (c) culturing the LBO in a three-dimensional (3D) matrix in the presence of a Wnt agonist (e.g., a GSK inhibitor such as CHIR99021), a BMP agonist (e.g., BMP4), one or more FGF agonists (e.g., FGF10, KGF) and retinoic acid (or its derivative), to form a branched LBO (BLBO); and (d) dissociating the LBO or BLBO, and culturing the dissociated LBO or BLBO in a 3D matrix in the presence of a Wnt agonist (e.g., a GSK inhibitor such as CHIR99021), an FGF agonist (e.g., KGF), a corticosteroid (e.g., dexamethasone), a 3′,5′-cyclic adenosine monophosphate (cAMP) pathway activator (e.g., cAMP, or 8-bromo-cAMP) and a phosphodiesterase (PDE) inhibitor (e.g., 3-isobutyl-1-methylxanthine).

In certain embodiments, in step (d) of the method, the dissociated LBO or BLBO is cultured to generate the lung progenitor cells.

In certain embodiments, the LBO or BLBO may be dissociated to single cells or cell clusters.

In certain embodiments, the dissociated LBO or BLBO may be cultured (e.g., step (d)) in the presence of one or more of CHIR 99021, KGF (FGF7), dexamethasone, cAMP (or 8-bromo-cAMP), and 3-isobutyl-1-methylxanthine. In one embodiment, CHIR 99021 is at a concentration of about 0.5 μM to about 10 μM, about 1 μM to about 8 μM, about 1 μM to about 5 μM, or about 3 μM. In one embodiment, KGF is at a concentration of about 1 ng/ml to about 50 ng/ml, about 2 ng/ml to about 30 ng/ml, about 5 ng/ml to about 15 ng/ml, or about 10 ng/ml. In one embodiment, dexamethasone is at a concentration of about 50 nM to about 500 nM, about 80 nM to about 300 nM, about 100 nM to about 200 nM, about 127 nM or 50 ng/ml. In one embodiment, cAMP (or 8-bromo-cAMP) is at a concentration of about 0.01 mM about 1 mM, about 0.05 mM about 0.8 mM, about 0.06 mM about 0.5 mM, or about 0.1 mM. In one embodiment, 3-isobutyl-1-methylxanthine is at a concentration of about 0.01 mM about 1 mM, about 0.05 mM about 0.8 mM, about 0.06 mM about 0.5 mM, or about 0.1 mM.

Anterior foregut endoderm cells are first generated from mammalian pluripotent stem cells (PSCs). In certain embodiments, PSCs (e.g., ESC or iPSC cells) are cultured in a cell culture medium containing Y-27632 and BMP4 (e.g., the primitive streak/embryoid body medium containing about 10 μM Y-27632, about 3 ng/ml BMP4) to allow embryoid body formation. Embryoid bodies are fed (e.g., every day) with fresh cell culture media containing Y-27632, BMP4, FGF2 and Activin A (e.g., the endoderm induction medium containing about 10 μM Y-27632, about 0.5 ng/ml BMP4, about 2.5 ng/ml FGF2 and about 100 ng/ml Activin A). Cells are then cultured in a cell culture medium containing Noggin and SB431542 (e.g., the anteriorization medium 1 containing about 100 ng/ml Noggin and about 10 μM SB431542) for a period of time (e.g., about 24 hours), followed by being cultured in a cell culture medium containing SB431542 and IWP2 (e.g., the anteriorization medium 2 containing about 10 μM SB431542 and about 1 μM IWP2) for a period of time (e.g., about 24 hours).

Lung bud organoids (LBOs) and branched LBOs (BLBOs) can be generated from anterior foregut endoderm cells. In certain embodiments, anterior foregut endoderm cells are cultured in a suspension culture (e.g., a branching medium) containing CHIR99021, FGF10, KGF, BMP4 and retinoic acid (e.g., the ventralization/branching medium containing about 3 μM CHIR99021, about 10 ng/ml FGF10, about 10 ng/ml KGF, about 10 ng/ml BMP4 and about 50 nM all-trans retinoic acid) for a period of time (e.g., about 48 hours) to form three-dimensional clumps which then fold into lung bud organoids or LBOs (e.g., as early as day 10 to day 12, with day 0 being the start of culturing the PSCs). The cell culture medium (e.g., the branching medium) is changed (e.g., regularly such as every other day until day 20 to day 25). The LBOs are embedded in a 3D matrix (e.g., about 50% to about 100%, at least or about 50%, at least or about 60%, at least or about 70%, at least or about 80%, at least or about 90%, or about 100%, 3D matrix such as Matrigel or any matrix as described herein). The cell culture medium containing CHIR99021, FGF10, KGF, BMP4 and retinoic acid (e.g., the branching medium containing about 3 μM CHIR99021, about 10 ng/ml FGF10, about 10 ng/ml KGF, about 10 ng/ml BMP4 and about 50 nM all-trans retinoic acid) is added after the 3D matrix solidifies to help generate branched LBOs (BLBOs).

As used herein, day 0 is the start of culturing the stem cells (e.g., PSCs), unless specifically stated otherwise.

Lung progenitor cells (e.g., transitional lung organoids (TLOs)) can be generated from LBOs or BLBOs. In certain embodiments, the 3D matrix (e.g., Matrigel) embedded LBOs or BLBOs can be used for lung progenitor cell generation (e.g., when they reach day 42 of development or later). The LBOs or BLBOs are released from the 3D matrix (e.g., Matrigel), and then dissociated, e.g., to single cells. The dissociated LBOs or BLBOs are then cultured in a 3D matrix (e.g., Matrigel), in the presence of a Wnt agonist (e.g., a GSK inhibitor such as CHIR99021), an FGF agonist (e.g., KGF), a corticosteroid (e.g., dexamethasone), a cAMP pathway activator (e.g., cAMP or 8-bromo-cAMP) and a phosphodiesterase (PDE) inhibitor (e.g., 3-isobutyl-1-methylxanthine) (e.g., in the presence of about 3 μM CHIR 99021, about 10 ng/ml KGF, about 50 ng/ml dexamethasone, about 0.1 mM 8-Bromoadenosine 3′,5′-cyclic monophosphate sodium salt and about 0.1 mM 3-isobutyl-1-methylxanthine). After a period of time (e.g., about 2 to about 3 weeks), a lung progenitor cell (e.g., TLO) culture is established that can be maintained by regular passaging for, e.g., six months or longer.

In certain embodiments, the present method does not require sorting for lineage-specific reporters or surface markers to enrich for desired lung lineages to isolate these prior to further differentiation.

The lung progenitor cells (e.g., lung organoids) may be serially passaged every (about) 1 week, every (about) 2 weeks, every (about) 3 weeks, every (about) 4 weeks, every (about) 5 weeks, every (about) 3 days, every (about) 4 days, every (about) 5 days, every (about) 6 days, every (about) 8 days, every (about) 9 days, every (about) 10 days, every (about) 11 days, every (about) 12 days, every (about) 13 days, every (about) 15 days, every (about) 16 days, every (about) 17 days, every (about) 18 days, every (about) 19 days, or every (about) 20 days. The lung progenitor cells (e.g., lung organoids) may be passaged for at least or about 1 passage, at least or about 2 passages, at least or about 3 passages, at least or about 4 passages, at least or about 5 passages, at least or about 6 passages, at least or about 7 passages, at least or about 8 passages, at least or about 9 passages, at least or about 10 passages, or more passages.

The lung progenitor cells (e.g., lung organoids) may be cultured for 1 or more passages, for example, 1, 2, 3, 4, 5, 6, 7, 8, 9, 10, 15, 20, 25, 30 or more passages, for example, 20-30 passages, 30-35 passages, 32-40 passages or more. In some embodiments, an expanding cell population or organoid is split/passaged once a month, once every two weeks, once a week, twice a week, three times a week, four times a week, five times a week, six times a week or daily.

The present disclosure also provides lung progenitor cells (e.g., lung organoids) which have been cultured for at least or about 1 week, at least or about 2 weeks, at least or about 3 weeks, at least or about 4 weeks, at least or about 5 weeks, at least or about 6 weeks, at least or about 7 weeks, at least or about 8 weeks, at least or about 9 weeks, at least or about 10 weeks, at least or about 12 weeks, at least or about 14 weeks, at least or about 16 weeks, at least or about 18 weeks, at least or about 20 weeks.

The cell culture medium used in the present method may be a serum-free medium or a serum-containing medium.

The lung progenitor cells may be cryopreserved, or thawed after cryopreservation.

The present disclosure provides for lung progenitor cells derived from mammalian pluripotent stem cells (e.g., human pluripotent stem cells or hPSCs), and methods for generating these cells. PSCs may comprise embryonic stem cells (ESCs) and/or induced pluripotent stem cells (iPSCs). Derived from the inner cell mass of the blastocyst, ESCs can be maintained in a pluripotent state in vitro and have the potential to generate every cell type in the organism. iPSCs may be generated by reprogramming somatic cells to a pluripotent state similar to ESCs, and can be patient-specific.

Producing anterior foregut endoderm cells from mammalian PSCs (e.g., step (a)), may last for about 2 days to about 8 days, about 3 days to about 7 days, about 3 days to about 6 days, about 3 days, or about 6 days. Producing anterior foregut endoderm cells from mammalian PSCs (e.g., step (a)) may be conducted, for example, at a time point ranging from day 3 to day 8, or from day 4 to day 6, counting from the start of culturing the PSCs (day 0).

Culturing the anterior foregut endoderm cells in a suspension culture to generate at least one lung bud organoid (LBO) (e.g., step (b)), may last for about 2 days to about 30 days, about 5 days to about 28 days, about 10 days to about 25 days, about 15 days to about 25 days, about 16 days to about 23 days, about 10 days to about 16 days, about 10 days to about 30 days, about 10 days to about 20 days, about 16 days, or about 23 days. Culturing the anterior foregut endoderm cells in a suspension culture to generate at least one lung bud organoid (LBO) (e.g., step (b)) may be conducted, for example, at a time point ranging from day 8 to day 30, or from day 10 to day 25, counting from the start of culturing the PSCs.

Embedding the LBO within a 3D matrix (e.g., in step (c)), may be conducted at a time point ranging from day 20 to day 30, or day 25, counting from the start of culturing the PSCs.

Culturing the embedded LBO to form branched LBO (BLBO) (e.g., in step (c)), may last for about 20 days to about 200 days, about 30 days to about 180 days, about 50 days to about 160 days, about 100 days to about 200 days, about 20 days to about 50 days, about 20 days to about 30 days, about 10 days to about 30 days, or about 10 days to about 20 days. Culturing the embedded LBO to form branched LBO (BLBO) (e.g., in step (c)) may be conducted, for example, at a time point ranging from day 20 to day 180, counting from the start of culturing the PSCs.

The LBO or BLBO may be dissociated (e.g., in step (d)) at a time point ranging from about day 20 to about day 180, or from about day 25 to about day 150, counting from the start of culturing the PSCs. The dissociated LBO or BLBO may be cultured (e.g., in step (d)) in a 3D matrix for desired time periods.

Stem cells (e.g., pluripotent stem cells, such as embryonic stem (ES) cells or induced pluripotent cells (iPSCs)) are subjected to a series of different culture steps to orchestrate differentiation of the stem cells into definitive endoderm (DE), anterior foregut endoderm (AFE) cells, and then into LBOs. In certain embodiments, LBOs (up to about 20-25 days in suspension culture) express sonic hedgehog (SHH) on the tips of budding epithelial structures but lack branching structures. The LBOs are then embedded in a 3D matrix (e.g., Matrigel). BLBOs contain mesoderm and pulmonary endoderm.

The phenotypes of the various cells may be:

1. Embryonic stem cells or iPSC cells: undifferentiated.
2. Definitive endoderm: FOXA2+, cKIT+, CXCR4+, EPCAM+(epithelial marker).
3. Anterior foregut endoderm: FOXA2+, SOX2+, EPCAM+, CDX2−.
4. Ventral anterior foregut endoderm: FOXA2+, NKX2.1+, EPCAM+.
5. Lung bud organoids: FOXA2+NKX2.1+EPCAM+.
6. The lung bud organoids may generate branching colonies after plating in a 3D matrix.

The lung bud organoids (LBOs) have the capacity of developing into branching airways and alveolar structures that at least partially recapitulate human lung development. Branched LBOs (BLBOs) contain pulmonary endoderm and mesoderm compatible with pulmonary mesenchyme, and undergo branching morphogenesis. They develop predominantly into structures compatible with distal lung, i.e., alveolar structures containing alveolar epithelial cells, but also contain some more proximal, i.e., airway cells.

In certain embodiments, development of the LBO may occur in three stages:

Stage 1: suspension culture of in vitro generated anterior foregut endoderm cells to form LBOs that are spherical structures with folded epithelium (up to day 25).
Stage 2: In a 3D matrix (e.g., Matrigel) culture, which starts at about day 20 to day 25, the unbranched LBO spheres start branching within one week.
Stage 3: When cultured in a 3D matrix (e.g., Matrigel), the BLBOs begin to show dilated tips which have the morphogenesis of alveolar structures.

In certain embodiments, first, stem cells (e.g., hPSCs) are subjected to the embryoid bodies/primitive streak formation medium under conditions to induce differentiation of the pluripotent cells to definitive endoderm (DE). This first stage typically takes 4 days (day 0 to day 4 counting from the start of culturing the stem cells) and forms embryoid bodies having endoderm (e.g., as determined through expression of CXCR4 and c-kit). Second, day 5 to day 6, embryoid bodies are subjected to the anteriorization medium under conditions for the embryoid bodies to form anterior foregut patterning. Third, day 6 to day 20-25, cells are then subjected to the ventralization medium/branching medium under conditions that induce ventralization and ultimate production of lung bud organoids (LBOs). LBO formation may be determined by sonic hedgehog (SHH) expression on the tips of budding epithelial structures. Upon production of LBOs between day 20-day 25 of the culture process, organoids that have folding structures are then selected and embedded into a 3D matrix (e.g., Matrigel) in a sandwich configuration. Folding structures include folding sheets of EPCAM+KRT8+ECAD+FOXA1/2+AFE cells (e.g., FOXA2: 89.07%±3.36%, EPCAM+: 92.08%±1.88%, n=3; RUES2 ESCs). Forming the sandwich involves adding a first amount of the 3D matrix (e.g., Matrigel) in a well or other suitable container and allowed to solidify to form the bottom portion of the sandwich. The selected organoids having folding structures are mixed with the 3D matrix (e.g., Matrigel) and placed on top of the bottom portion and allowed to solidify to form the center cell layer. Another amount of the 3D matrix (e.g., Matrigel) without cells is placed on top of the embedded cell layer and allowed to solidify to form the top portion of the sandwich. Ventralization media/Branching media is placed in the well and replenished periodically. Generation of branching buds from organoids may occur one week after embedding into the 3D matrix (e.g., Matrigel). Extensive branching organoids may appear about 2-3 weeks post embedding. The longer the LBO is cultured (e.g., in a 3D matrix) the more developed the branching morphogenesis is. BLBO cultures may be grown for 180 days or longer. The longer the BLBOs are grown, the more mature alveolar cells and the larger the organoids.

In certain embodiments, after embedding day 25 LBOs from RUES2 in Matrigel in the presence of CHIR99021, FGF10, FGF7, BMP4 and RA, >95% yield rapidly expanding branching structures (e.g., starting from RUES2, iPSCs, including C12, a line from a patient with mutations IRF7 that causes acute respiratory distress syndrome after influenza infection). The RUES2 cells express markers of pulmonary endoderm (e.g., FOXA2+: 95.17%±1.54%, NKX2.1+: 74.97%±4.37%, EPCAM+: 96.83%±0.62%, SOX9+: 92.42%±3.81%, n=3 at d70; RUES2 ESCs). Uniform luminal expression of MUC1 demonstrates polarization. Cells expressing the ATII markers SFTPC, SFTPB and ABCA3 are present in all structures.

Such methods are not limited to a particular manner of accomplishing the directed differentiation of stem cells (e.g., PSCs) into anterior foregut endoderm cells. Indeed, any suitable method for producing anterior foregut endoderm cells from stem cells such as pluripotent stem cells (e.g., iPSCs or ESCs) is applicable to the methods described herein.

The present disclosure also provides for lung progenitor cells generated by the present methods, or a cell population comprising the lung progenitor cells generated by the present methods. The cell population may be in the form of a lung organoid.

The present disclosure provides for an artificial lung organoid, lung progenitor cells, or a cell population generated in vitro. The cell population may be in the form of a lung organoid.

The artificial lung organoid, lung progenitor cells, or cell population may comprise type 0 alveolar epithelial (AT0) cells, terminal respiratory bronchiole stem cells (TRB-SCs) and distal basal cells (BCs). The three-dimensional artificial lung construct, lung progenitor cells, or cell population may further comprise neuroendocrine cells.

In certain embodiments, at least or about 10%, at least or about 20%, at least or about 30%, at least or about 40%, at least or about 50%, at least or about 60%, at least or about 70%, at least or about 80%, at least or about 90%, of the artificial lung organoid, lung progenitor cells, or cell population are AT0 cells. In certain embodiments, at least or about 10%, at least or about 20%, at least or about 30%, at least or about 40%, at least or about 50%, at least or about 60%, at least or about 70%, at least or about 80%, at least or about 90%, of the artificial lung organoid, lung progenitor cells, or cell population are TRB-SCs. In certain embodiments, at least or about 10%, at least or about 20%, at least or about 30%, at least or about 40%, at least or about 50%, at least or about 60%, at least or about 70%, at least or about 80%, at least or about 90%, of the artificial lung organoid, lung progenitor cells, or cell population are distal BCs.

The three-dimensional artificial lung construct, lung progenitor cells, or cell population may comprise SCGB3A2⁺SFTPB⁺SFTPC⁺ cells, SFTPB⁺SCGB3A2⁺SFTPC⁻ cells, and SFPTB⁺SCGB3A2⁻ cells.

In certain embodiments, at least or about 10%, at least or about 20%, at least or about 30%, at least or about 40%, at least or about 50%, at least or about 60%, at least or about 70%, at least or about 80%, at least or about 90%, of the artificial lung organoid, lung progenitor cells, or cell population are SCGB3A2⁺SFTPB⁺SFTPC⁺. In one embodiment, at least or about 70% of the artificial lung organoid, lung progenitor cells, or cell population are SCGB3A2⁺SFTPB⁺SFTPC⁺.

In certain embodiments, at least or about 10%, at least or about 20%, at least or about 30%, at least or about 40%, at least or about 50%, at least or about 60%, at least or about 70%, at least or about 80%, at least or about 90%, of the artificial lung organoid, lung progenitor cells, or cell population are SFTPB⁺SCGB3A2⁺SFTPC⁻. In one embodiment, at least or about 30% of the artificial lung organoid, lung progenitor cells, or cell population are SFTPB⁺SCGB3A2⁺SFTPC⁻.

In certain embodiments, at least or about 10%, at least or about 20%, at least or about 30%, at least or about 40%, at least or about 50%, at least or about 60%, at least or about 70%, at least or about 80%, at least or about 90%, of the artificial lung organoid, lung progenitor cells, or cell population are SFPTB⁺SCGB3A2⁻. In one embodiment, at least or about 30% of the artificial lung organoid, lung progenitor cells, or cell population are SFPTB⁺SCGB3A2⁻.

The artificial lung organoid, lung progenitor cells, or cell population may express SFTPC and SFTPB. The artificial lung organoid, lung progenitor cells, or cell population may express EPCAM, NKX2.1, SFTPB, or combinations thereof. The artificial lung organoid, lung progenitor cells, or cell population may further express SCGB3A2. The artificial lung organoid, lung progenitor cells, or cell population may express mRNAs encoding SCGB3A2, SFTPC, SFTPB, ABCA3, LPCAT, NAPSA, SLC234A2, LAMP3, or combinations thereof.

The present disclosure provides for a pharmaceutical composition comprising the present lung progenitor cells, cell population, or artificial lung organoid.

The pharmaceutical composition may further comprise a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. Exemplary physiologically acceptable carriers include sterile, pyrogen-free water and sterile, pyrogen-free, phosphate buffered saline.

The lung organoid, cell population, cell aggregates, or cell clusters may be dissociated by an enzymatic treatment. For example, the enzyme(s) may comprise at least one protease. The organoid, cell aggregates, or cell clusters may be dissociated by dispase, accutase, trypsin, and/or collagenase (e.g., collagenase I, II, III, and IV, etc.).

The present lung progenitor cells, cell population, artificial lung organoid, or pharmaceutical composition may be engrafted, transplanted, or implanted into a subject. The present lung progenitor cells, cell population, artificial lung organoid, or pharmaceutical composition may be administered to the subject by routes including, but not limited to, intranasal, direct delivery to a desired tissue/organ (e.g., the lung, airway or nasal cavity of a subject), oral, inhalation, intratracheal, intravenous, intramuscular, subcutaneous, intradermal, and other parental routes of administration. Additionally, routes of administration may be combined, if desired.

The lung progenitor cells may be non-syngeneic with the subject. The lung progenitor cells may be syngeneic with the subject. The lung progenitor cells may be allogeneic or xenogeneic with the subject. The lung progenitor cells may be autologous or allogeneic to the subject.

The present disclosure provides for a biological scaffold comprising the present lung progenitor cells.

Also encompassed by the present disclosure is a method of treating a pulmonary disorder or injury in a subject in need thereof. The method may comprise administering to the subject an effective amount (e.g., a therapeutically effective amount) of the present lung progenitor cells, artificial lung organoid, or pharmaceutical composition.

The method may comprise engrafting an effective amount (e.g., a therapeutically effective amount) of the present lung progenitor cells, artificial lung organoid, or pharmaceutical composition into the lung, airway or nasal cavity of the subject. The engrafted cells may integrate into the epithelium.

The present disclosure provides for methods of using the present lung progenitor cells, cell population, or artificial lung organoid in a drug discovery screen; toxicity assay; research of tissue embryology, cell lineages, and differentiation pathways; gene expression studies including recombinant gene expression; research of mechanisms involved in tissue injury and repair; research of inflammatory and infectious diseases; studies of pathogenetic mechanisms; or studies of mechanisms of cell transformation and etiology of cancer.

A Wnt agonist, or an agonist (or activator) of the Wnt signaling, may be used in one or more of the following steps: culturing the anterior foregut endoderm cells in a suspension culture to generate at least one lung bud organoid (LBO) (e.g., step (b)); culturing the LBO embedded in a 3D matrix to form a branched LBO (BLBO) (e.g., step (c)); and culturing the dissociated LBO or BLBO in a 3D matrix (e.g., step (d)).

The “Wnt signaling activator” or “Wnt signaling agonist” as used herein refers to an agent that activates the Wnt signaling pathway.

The Wnt signaling pathway can include a series of events that occur when a Wnt protein binds to a cell-surface receptor of a Frizzled receptor family member. This results in the activation of Dishevelled family proteins which inhibit a complex of proteins that includes axin, GSK-3, and the protein APC to degrade intracellular β-catenin. The resulting enriched nuclear β-catenin enhances transcription by TCF/LEF family transcription factors.

A Wnt agonist (e.g., a small molecule or other agents) may be an agent that activates TCF/LEF-mediated transcription in a cell. A Wnt agonist can be an agent that binds and activates a Frizzled receptor family member including any and all of the Wnt family proteins, an inhibitor of intracellular β-catenin degradation, and an activator of TCF/LEF. The Wnt agonist may stimulate a Wnt activity in a cell by at least or about 10%, at least or about 20%, at least or about 30%, at least or about 50%, at least or about 70%, at least or about 90%, at least or about 100%, relative to a level of the Wnt activity in the absence of said agent, as assessed in the same cell type. As known to a skilled person, a Wnt activity can be determined by measuring the transcriptional activity of Wnt, for example by pTOPFLASH and pFOPFLASH Tcfluciferase reporter constructs (see, e.g., Korinek et al., 1997, Science 275:1784-1787).

A Wnt agonist may comprise a secreted glycoprotein including Wnt-1/Int-1; Wnt-2/Irp (Int-1-related Protein); Wnt-2b/13; Wnt-3/Int-4; Wnt-3a; Wnt-4; Wnt-5a; Wnt-5b; Wnt-6 (Kirikoshi H et al. 2001, Biochem. Biophys. Res. Com. 283: 798-805); Wnt-7a; Wnt-7b; Wnt-8a/8d; Wnt-8b; Wnt-9a/14; Wnt-9b/14b/15; Wnt-10a; Wnt-10b/12; Wnt-11; and Wnt-16.

Further Wnt agonists include the R-spondin family of secreted proteins, which is implicated in the activation and regulation of Wnt signaling pathway and which is comprised of 4 members (R-spondin 1 (NU206, Nuvelo, San Carlos, Calif.), R-spondin 2, R-spondin 3, and R-spondin-4); and Norrin (also called Norrie Disease Protein or NDP), which is a secreted regulatory protein that functions like a Wnt protein in that it binds with high affinity to the Frizzled-4 receptor and induces activation of the Wnt signaling pathway (Kestutis Planutis et al. (2007) BMC Cell Biol. 8: 12). Compounds that mimic the activity of R-spondin may be used as Wnt agonists. Lgr5 agonists such as agonistic anti-Lgr5 antibodies are examples of Wnt agonists that may be used.

A small molecule agonist of the Wnt signaling pathway, an aminopyrimidine derivative, may also be used as a Wnt agonist (Liu et al. (2005) Angew Chem Int Ed Engl. 44, 1987-90).

Examples of the Wnt signaling activator include glycogen synthase kinase (GSK) inhibitors such as GSK3 inhibitors. In some embodiments, activation of Wnt/beta-catenin signaling is achieved by inhibiting GSK3 phosphotransferase activity or GSK3 binding interactions. GSK3 inhibition can be achieved in a variety of ways including, but not limited to, providing small molecules that inhibit GSK3 phosphotransferase activity, RNA interference (RNAi such as small interfering RNAs or siRNAs, and short hairpin RNAs or shRNAs) against GSK3, and overexpression of dominant negative form of GSK3. Dominant negative forms of GSK3 are known in the art as described, e.g., in Hagen et al. (2002), J. Biol. Chem., 277(26):23330-23335, which describes a Gsk3 comprising an R96A mutation.

In some embodiments, GSK3 is inhibited by contacting a cell with a small molecule that inhibits GSK3 phosphotransferase activity or GSK3 binding interactions. Suitable small molecule Gsk3 inhibitors include, but are not limited to, CHIR99021, CHIR98014, BIO-acetoxime, 6-Bromoindirubin-3′-oxime (BIO), LiCl, SB 216763, SB 415286, AR A014418, Kenpaullone, 1-Azakenpaullone, Bis-7-indolylmaleimide, TWS119, and any combinations thereof.

GSK3 inhibitors also include lithium, and FRAT-family members and FRAT-derived peptides that prevent interaction of GSK3 with axin. An overview is provided by Meijer et al., (2004) Trends in Pharmacological Sciences 25, 471-480. Methods and assays for determining a level of GSK3 inhibition are known to a skilled person and comprise, for example, the methods and assay as described in Liao et al 2004, Endocrinology, 145(6): 2941-9.

In certain embodiments, the GSK3 activity may be inhibited by RNA interference targeting GSK3. For example, GSK3 expression levels can be knocked-down using siRNAs against GSK3, or a retroviral vector with an inducible expression cassette for GSK3, e.g., a Tet-inducible retroviral RNA interference (RNAi) system, or a cumate-inducible system.

In some embodiments, an agonist of Wnt signaling is Wnt3a, which mediates canonical Wnt signaling; any inducer of canonical Wnt signaling can be used, including, for example, Wnt/beta-catenin pathway agonists glycogen synthase kinase 3 beta (GSK3b) inhibitors, and casein kinase 1 (CK1) inhibitors.

Non-limiting examples of Wnt agonists include DNA encoding β-catenin (e.g., vectors encoding β-catenin, etc.), β-catenin polypeptides, one or more Wnt/β-catenin pathway agonists (e.g., Wnt ligands, DSH/DVL-1, -2, -3, LRP6N, WNT3A, WNT5A, and WNT3A), one or more glycogen synthase kinase 33 (GSK30) inhibitors (e.g., lithium chloride (LiCl), Purvalanol A, olomoucine, alsterpaullone, kenpaullone, benzyl-2-methyl-1,2,4-thiadiazolidine-3,5-dione (TDZD-8), 2-thio(3-iodobenzyl)-5-(1-pyridyl)-[1,3,4]-oxadiazole, 2,4-dibenzyl-5-oxothiadiazolidine-3-thione (OTDZT), (2′Z,3′E)-6-Bromoindirubin-3′-oxime (BIO), α-4-Dibromoacetophenone (Tau Protein Kinase I (TPK I) Inhibitor), 2-Chloro-1-(4,5-dibromo-thiophen-2-yl)-ethanone, N-(4-Methoxybenzyl)-N′-(5-nitro-1,3-thiazol-2-yl)urea (AR-A014418), indirubin-5-sulfonamide; indirubin-5-sulfonic acid (2-hydroxyethyl)-amide indirubin-3′-monoxime; 5-iodo-indirubin-3′-monoxime; 5-fluoroindirubin; 5,5′-dibromoindirubin; 5-nitroindirubin; 5-chloroindirubin; 5-methylindirubin, 5-bromoindirubin, 4-Benzyl-2-methyl-1,2,4-thiadiazolidine-3,5-dione (TDZD-8), H-KEAPPAPPQSpP-NH2 (L803) and Myr-N-GKEAPPAPPOSpP-NH2 (L803-mts)), one or more anti-sense RNA or siRNA that bind specifically to GSK3, one or more casein kinase 1 (CK1) inhibitors (e.g., antisense RNA or siRNA that binds specifically to CK1 mRNA), protease inhibitors, and proteasome inhibitors.

In certain embodiments, the GSK3 inhibitor (e.g., CHIR99021 or any agent described herein) is used at a concentration ranging from about 1 μM to about 100 μM, from about 1 μM to about 30 μM, from about 1 μM to about 20 μM, at least or about 1 μM, at least or about 2 μM, at least or about 3 μM, at least or about 4 μM, at least or about 5 μM, at least or about 6 μM, at least or about 7 μM, at least or about 8 μM, at least or about 9 μM, at least or about 10 μM, at least or about 11 μM, at least or about 12 μM, at least or about 13 μM, at least or about 14 μM, at least or about 15 μM, at least or about 16 μM, at least or about 17 μM, at least or about 18 μM, at least or about 19 μM, or at least or about 20 μM, or higher concentrations. In another embodiment, the GSK3 inhibitor is used at a concentration ranging from about 0.1 μM to about 1 μM, e.g., at least or about 0.1 μM, at least or about 0.2 μM, at least or about 0.3 μM, at least or about 0.4 μM, at least or about 0.5 μM, at least or about 0.6 μM, at least or about 0.7 μM, at least or about 0.8 μM, at least or about 0.9 μM, or at least or about 1 μM.

An FGF agonist, or an agonist (or activator) of the FGF signaling, may be used in one or more of the following steps: culturing the anterior foregut endoderm cells in a suspension culture to generate at least one lung bud organoid (LBO) (e.g., step (b)); culturing the LBO embedded in a 3D matrix to form a branched LBO (BLBO) (e.g., step (c)); and culturing the dissociated LBO or BLBO in a 3D matrix (e.g., step (d)).

The agonists of the FGF signaling include, but are not limited to, FGF7 or keratinocyte growth factor (KGF), FGF9, or FGF10. In some embodiments, other agonists of FGF signaling can be used, e.g., FGF1, FGF2, FGF3, FGF5, FGF6, FGF9, FGF11, FGF12, FGF13, FGF14, FGF15, FGF16, FGF17, FGF18, FGF19, FGF20, FGF21, FGF22, or FGF23.

For example, FGF (e.g., FGF7 or FGF10 or any FGF as described herein) may be at a concentration of about 1 ng/ml to 10 g/ml, 10 ng/ml to 1 g/ml, 10 ng/ml to 500 ng/ml, 10 ng/ml to 250 ng/ml, 10 ng/ml to 100 ng/ml, at least or about 1 ng/ml, at least or about 2 ng/ml, at least or about 3 ng/ml, at least or about 4 ng/ml, at least or about 5 ng/ml, at least or about 6 ng/ml, at least or about 7 ng/ml, at least or about 8 ng/ml, at least or about 9 ng/ml, at least or about 10 ng/ml, at least or about 11 ng/ml, at least or about 12 ng/ml, at least or about 13 ng/ml, at least or about 14 ng/ml, at least or about 15 ng/ml, at least or about 16 ng/ml, at least or about 17 ng/ml, at least or about 18 ng/ml, at least or about 19 ng/ml, at least or about 20 ng/ml, at least or about 25 ng/ml, at least or about 30 ng/ml, at least or about 35 ng/ml, at least or about 40 ng/ml, at least or about 45 ng/ml, at least or about 50 ng/ml, at least or about 55 ng/ml, at least or about 60 ng/ml, at least or about 65 ng/ml, at least or about 70 ng/ml, at least or about 75 ng/ml, at least or about 80 ng/ml, at least or about 85 ng/ml, at least or about 90 ng/ml, at least or about 95 ng/ml, or at least or about 100 ng/ml. In certain embodiments, FGF7 and/or FGF10 is/are at a concentration of about 25 ng/ml to 150 ng/ml, 50 ng/ml to 150 ng/ml, or 75 ng/ml to 150 ng/ml. In certain embodiments, FGF7 and/or FGF10 are present at a concentration of about 10 ng/ml.

The dissociated LBO or BLBO may be cultured in a 3D matrix (e.g., step (d)) in the presence of a steroid (e.g., a corticosteroid). The steroid may be a glucocorticoid or a mineralocorticoid. Exemplary steroids include, but are not limited to, dexamethasone, dexamethasone derivatives, beclometasone, betamethasone, fluocortolone, halometasone, mometasone, prednisone, prednisone derivatives, fludrocortisone, hydrocortisone (cortisol), hydrocortisone acetate, cortisone acetate, tixocortol pivalate, prednisolone, methylprednisolone, corticosterone, cortisone, aldosterone, amcinonide, budesonide, desonide, fluocinolone acetonide, fluocinonide, halcinonide, triamcinolone acetonide, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate.

The concentration of the corticosteroid (e.g., dexamethasone or any steroid as described herein) may range from about 10 nM to about 10 μM, from about 10 nM to about 5 μM, from about 10 nM to about 1 μM, from about 10 nM to about 500 nM, from about 20 nM to about 400 nM, from about 30 nM to about 300 nM, from about 40 nM to about 250 nM, from about 50 nM to about 200 nM, from about 50 nM to about 180 nM, from about 50 nM to about 160 nM, from about 50 nM to about 150 nM, from about 50 nM to about 130 nM, from about 60 nM to about 300 nM, from about 60 nM to about 250 nM, from about 60 nM to about 200 nM, from about 60 nM to about 180 nM, from about 60 nM to about 160 nM, from about 60 nM to about 150 nM, from about 60 nM to about 130 nM, from about 80 nM to about 300 nM, from about 80 nM to about 250 nM, from about 80 nM to about 200 nM, from about 80 nM to about 180 nM, from about 80 nM to about 160 nM, from about 80 nM to about 150 nM, from about 80 nM to about 130 nM, from about 100 nM to about 300 nM, from about 100 nM to about 250 nM, from about 100 nM to about 200 nM, from about 100 nM to about 180 nM, from about 100 nM to about 160 nM, from about 100 nM to about 150 nM, from about 100 nM to about 130 nM, at least or about 10 nM, at least or about 20 nM, at least or about 30 nM, at least or about 40 nM, at least or about 50 nM, at least or about 60 nM, at least or about 70 nM, at least or about 80 nM, at least or about 90 nM, at least or about 100 nM, at least or about 110 nM, at least or about 120 nM, at least or about 125 nM, at least or about 127 nM, at least or about 130 nM, at least or about 140 nM, at least or about 150 nM, at least or about 15 nM, at least or about 25 nM, at least or about 35 nM, at least or about 45 nM, at least or about 55 nM, at least or about 65 nM, at least or about 75 nM, at least or about 85 nM, at least or about 95 nM, or at least or about 5 nM.

The concentration of the corticosteroid (e.g., dexamethasone or any steroid as described herein) may range from about 10 ng/ml to about 100 ng/ml, from about 10 ng/ml to about 90 ng/ml, from about 10 ng/ml to about 80 ng/ml, from about 10 ng/ml to about 70 ng/ml, from about 10 ng/ml to about 60 ng/ml, from about 10 ng/ml to about 50 ng/ml, from about 20 ng/ml to about 100 ng/ml, from about 20 ng/ml to about 90 ng/ml, from about 20 ng/ml to about 80 ng/ml, from about 20 ng/ml to about 70 ng/ml, from about 20 ng/ml to about 60 ng/ml, from about 20 ng/ml to about 50 ng/ml, from about 30 ng/ml to about 100 ng/ml, from about 30 ng/ml to about 90 ng/ml, from about 30 ng/ml to about 80 ng/ml, from about 30 ng/ml to about 70 ng/ml, from about 30 ng/ml to about 60 ng/ml, from about 30 ng/ml to about 50 ng/ml, from about 40 ng/ml to about 100 ng/ml, from about 40 ng/ml to about 90 ng/ml, from about 40 ng/ml to about 80 ng/ml, from about 40 ng/ml to about 70 ng/ml, from about 40 ng/ml to about 60 ng/ml, from about 40 ng/ml to about 50 ng/ml, at least or about 10 ng/ml, at least or about 20 ng/ml, at least or about 30 ng/ml, at least or about 40 ng/ml, at least or about 50 ng/ml, at least or about 60 ng/ml, at least or about 70 ng/ml, at least or about 80 ng/ml, at least or about 90 ng/ml, or at least or about 100 ng/ml.

The dissociated LBO or BLBO may be cultured in a 3D matrix in the presence of a cAMP pathway activator. The cAMP pathway activator may be any suitable activator which increases the levels of cAMP in a cell. The cAMP pathway involves activation of many types of hormones and neurotransmitter G-protein coupled receptors. Binding of the hormone or neurotransmitter to its membrane-bound receptor induces a conformational change in the receptor that leads to activation of the α-subunit of the G-protein. The activated G subunit stimulates, while the non-activated G subunit inhibits, adenylyl cyclase. Stimulation of adenylyl cyclase catalyzes the conversion of cytoplasmic ATP to cAMP, thus increasing the levels of cAMP in the cell.

The cAMP pathway activator may be, for example, an adenylyl cyclase activator. Examples of suitable adenylyl cyclase activators include forskolin, a forskolin analogue and cholera toxin. In some embodiments, the cAMP pathway activator is forskolin. In some embodiments, the cAMP pathway activator is not cholera toxin. In some embodiments the cAMP pathway activator may be a cAMP analog, for example 8-bromo-cAMP. 8-bromo-cAMP is a cell-permeable cAMP analog having greater resistance to hydrolysis by phosphodiesterases than cAMP. In some embodiments, the cAMP pathway activator is NKH477.

In some embodiments, the cAMP pathway activator (e.g., cAMP, 8-bromo-cAMP, or any other cAMP pathway activator as described herein) is used at a concentration ranging from about 10 nM to about 500 μM, from about 10 nM to about 100 μM, from about 1 μM to about 50 μM, from about 1 μM to about 25 μM, from about 5 μM to about 1000 μM, from about 5 μM to about 500 μM, from about 5 μM to about 100 μM, from about 5 μM to about 50 μM, from about 5 M to about 25 μM, from about 10 μM to about 1000 μM, from about 10 μM to about 500 μM, from about 10 μM to about 100 μM, from about 10 μM to about 50 μM, from about 10 μM to about 25 M, from about 10 μM to about 1 mM, from about 10 μM to about 900 μM, from about 10 μM to about 800 μM, from about 10 μM to about 700 μM, from about 10 μM to about 600 μM, from about 10 μM to about 500 μM, from about 10 μM to about 400 μM, from about 10 μM to about 300 μM, from about 10 μM to about 200 μM, from about 10 μM to about 100 μM, from about 50 M to about 1 mM, from about 50 μM to about 900 μM, from about 50 μM to about 800 μM, from about 50 μM to about 700 μM, from about 50 μM to about 600 μM, from about 50 μM to about 500 μM, from about 50 μM to about 400 μM, from about 50 μM to about 300 μM, from about 50 μM to about 200 μM, or from about 50 μM to about 100 μM. In some embodiments the cAMP pathway activator is used at a concentration of at least or about 10 nM, at least or about 20 nM, at least or about 50 nM, at least or about 100 nM, at least or about 200 nM, at least or about 500 nM, at least or about 1 μM, at least or about 2 μM, at least or about 5 μM, at least or about 10 μM, at least or about 20 μM, at least or about 30 μM, at least or about 40 μM, at least or about 50 μM, at least or about 60 μM, at least or about 70 μM, at least or about 80 μM, at least or about 90 μM, at least or about 110 μM, at least or about 120 μM, at least or about 130 μM, at least or about 140 μM, at least or about 150 μM, at least or about 160 μM, or at least or about 100 μM (0.1 mM).

For example, NKH477 can in some embodiments be used at a concentration of between about 100 nM and about 10 μM, or at a concentration of about 100 nM, about 1 μM or about 10 μM. Forskolin can in some embodiments be used at a concentration of between about 1 μM and about 100 μM, or at a concentration of about 1 μM, about 10 μM or about 100 μM.

Cholera toxin can in some embodiments be used at a concentration of between about 1 ng/ml and about 500 ng/ml, about 10 ng/ml and about 100 ng/ml, about 50 ng/ml and about 100 ng/ml, or about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 200 ng/ml, about 300 ng/ml, about 400 ng/ml or about 500 ng/ml.

The dissociated LBO or BLBO may be cultured in a 3D matrix in the presence of a phosphodiesterase (PDE) inhibitor. The phosphodiesterase inhibitor may be a nonselective phosphodiesterase inhibitor or a selective phosphodiesterase inhibitor. The phosphodiesterase inhibitor may be a methylated xanthine or its derivatives. The phosphodiesterase inhibitors may be 3-isobutyl-1-methylxanthine (IBMX), caffeine, aminophylline, paraxanthine, pentoxifylline, theobromine, or theophylline. The phosphodiesterase inhibitors may be PDE4 inhibitors, including, but not limited to, mesembrenone, rolipram, ibudilast, piclamilast, luteolin, drotaverine, roflumilast, apremilast, and crisaborole. The phosphodiesterase inhibitors may be PDE10 inhibitors, including, but not limited to, papaverine (an opium alkaloid).

In some embodiments, the phosphodiesterase (PDE) inhibitor (e.g., 3-isobutyl-1-methylxanthine or any other PDE inhibitor as described herein) is used at a concentration ranging from about 10 nM to about 500 μM, from about 10 nM to about 100 μM, from about 1 μM to about 50 μM, from about 1 μM to about 25 μM, from about 5 μM to about 1000 μM, from about 5 M to about 500 μM, from about 5 μM to about 100 μM, from about 5 μM to about 50 μM, from about M to about 25 μM, from about 10 μM to about 1000 μM, from about 10 μM to about 500 μM, from about 10 μM to about 100 μM, from about 10 μM to about 50 μM, from about 10 μM to about 25 μM, from about 10 μM to about 1 mM, from about 10 μM to about 900 μM, from about M to about 800 μM, from about 10 μM to about 700 μM, from about 10 μM to about 600 μM, from about 10 μM to about 500 μM, from about 10 μM to about 400 μM, from about 10 μM to about 300 μM, from about 10 μM to about 200 μM, from about 10 μM to about 100 μM, from about 50 μM to about 1 mM, from about 50 μM to about 900 μM, from about 50 μM to about 800 M, from about 50 μM to about 700 μM, from about 50 μM to about 600 μM, from about 50 M to about 500 μM, from about 50 μM to about 400 μM, from about 50 μM to about 300 μM, from about 50 μM to about 200 μM, or from about 50 μM to about 100 μM. In some embodiments the PDE inhibitor is used at a concentration of at least or about 10 nM, at least or about 20 nM, at least or about 50 nM, at least or about 100 nM, at least or about 200 nM, at least or about 500 nM, at least or about 1 μM, at least or about 2 μM, at least or about 5 μM, at least or about 10 μM, at least or about 20 μM, at least or about 30 μM, at least or about 40 μM, at least or about 50 μM, at least or about 60 μM, at least or about 70 μM, at least or about 80 μM, at least or about 90 μM, at least or about 110 μM, at least or about 120 μM, or at least or about 100 μM (0.1 mM).

A BMP agonist, or an agonist (or activator) of the BMP signaling, may be used in one or more of the following steps: culturing the anterior foregut endoderm cells in a suspension culture to generate at least one lung bud organoid (LBO) (e.g., step (b)); and culturing the LBO embedded in a 3D matrix to form a branched LBO (BLBO) (e.g., step (c)).

The agonists of BMP signaling include, but are not limited to, BMP1, BMP2, BMP3, BMP4, BMP5, BMP6, BMP7, BMP8, BMP9, BMP10, BMP11, BMP12, BMP13, BMP14, BMP15, BMP16, BMP17, BMP18, BMP19, or BMP20. In certain embodiments, any of BMP 2-7 is/are used.

BMP may be present at a concentration of about 1 ng/ml to 10 g/ml, 10 ng/ml to 1 g/ml, 10 ng/ml to 500 ng/ml, 10 ng/ml to 250 ng/ml, or 10 ng/ml to 100 ng/ml. In certain embodiments, BMP4 is present at a concentration of about 25 ng/ml to 150 ng/ml, 50 ng/ml to 150 ng/ml or 75 ng/ml to 150 ng/ml. In certain embodiments, one or more BMP is/are present in cultures at a concentration of about 0.5 ng/ml, about 3 ng/ml, and/or about 10 ng/ml. For example, BMP (e.g., BMP4 or any BMP as described herein) may be present at a concentration of about 1 ng/ml to 10 g/ml, 10 ng/ml to 1 μg/ml, 10 ng/ml to 500 ng/ml, 10 ng/ml to 250 ng/ml, 10 ng/ml to 100 ng/ml, at least or about 1 ng/ml, at least or about 2 ng/ml, at least or about 3 ng/ml, at least or about 4 ng/ml, at least or about 5 ng/ml, at least or about 6 ng/ml, at least or about 7 ng/ml, at least or about 8 ng/ml, at least or about 9 ng/ml, at least or about 10 ng/ml, at least or about 11 ng/ml, at least or about 12 ng/ml, at least or about 13 ng/ml, at least or about 14 ng/ml, at least or about 15 ng/ml, at least or about 16 ng/ml, at least or about 17 ng/ml, at least or about 18 ng/ml, at least or about 19 ng/ml, at least or about 20 ng/ml, at least or about 25 ng/ml, at least or about 30 ng/ml, at least or about 35 ng/ml, at least or about 40 ng/ml, at least or about 45 ng/ml, at least or about 50 ng/ml, at least or about 55 ng/ml, at least or about 60 ng/ml, at least or about 65 ng/ml, at least or about 70 ng/ml, at least or about 75 ng/ml, at least or about 80 ng/ml, at least or about 85 ng/ml, at least or about 90 ng/ml, at least or about 95 ng/ml, or at least or about 100 ng/ml. In certain embodiments, BMP (e.g., BMP4 or any BMP as described herein) is present at a concentration of about 25 ng/ml to 150 ng/ml, 50 ng/ml to 150 ng/ml, or 75 ng/ml to 150 ng/ml. In certain embodiments, BMP (e.g., BMP4 or any BMP as described herein) is present at a concentration of about 10 ng/ml.

Retinoid acid, or its derivatives, may be used in one or more of the following steps: culturing the anterior foregut endoderm cells in a suspension culture to generate at least one lung bud organoid (LBO) (e.g., step (b)); and culturing the LBO embedded in a 3D matrix to form a branched LBO (BLBO) (e.g., step (c)). Retinoic acid may be all-trans retinoic acid, 9-cis retinoic acid, 13-cis retinoic acid, etc.

In certain embodiments, retinoic acid or its derivative is used at a concentration ranging from about 1 nM to about 100 nM, from about 20 nM to about 80 nM, from about 30 nM to about 60 nM, at least or about 10 nM, at least or about 20 μM, at least or about 30 nM, at least or about 40 nM, at least or about 50 nM, at least or about 60 nM, at least or about 70 nM, at least or about 80 nM, at least or about 90 nM, at least or about 100 nM, at least or about 15 nM, at least or about 25 nM, at least or about 35 nM, at least or about 45 nM, at least or about 55 nM, at least or about 65 nM, at least or about 75 nM, at least or about 85 nM, at least or about 95 nM, or at least or about 5 nM, or higher concentrations. In another embodiment, retinoic acid is used at a concentration ranging from about 40 nM to about 60 nM, e.g., at least or about 30 nM, at least or about 70 nM, at least or about 41 nM, at least or about 42 nM, at least or about 43 nM, at least or about 44 nM, at least or about 46 nM, at least or about 47 nM, at least or about 48 nM, or at least or about 49 nM.

The 3D (three-dimensional) matrix may include one or more extracellular matrix (ECM) proteins. The 3D matrices may include, but are not limited to, a solubilized basement membrane preparation from the Engelbreth-Holm-Swarm (EHS) mouse sarcoma, Matrigel, fibronectin, collagen (e.g., collagen I, collagen IV, etc.), collagen derivatives, gelatin, laminin, heparan sulfate proteoglycans, entactin/nidogen, cellulose, cellulose derivatives, cellulose polymers, proteoglycans, heparin sulfate, chondroitin sulfate, keratin sulfates, hyaluronic acid, elastin, fibrin, chitosan, alginate, vinculin, agar, agarose, hyaluronic acid, and combinations thereof. The 3D matrix may comprise one or more polymers including, but not limited to: polyethylene-imine and dextran sulfate, poly(vinylsiloxane)ecopolymerepoly-ethyleneimine, phosphorylcholine, poly(ethylene glycol), poly(lactic-glycolic acid), poly(lactic acid), polyhydroxyvalerte and copolymers, polyhydroxybutyrate and copolymers, polydiaxanone, polyanhydrides, polypeptides, poly(orthoesters), polyesters, and combinations thereof. The 3D matrix may comprise one or more matrices described in Gjorevsky et al, Nature, 2016, 539(7630):560-564 and DiMarco et al., Biomater Sci. 2015, 3(10):1376-85.

In one embodiment, the 3D matrix may comprise a gelatinous extracellular protein mixture secreted by Engelbreth-Holm-Swarm (EHS) mouse sarcoma cells. In one embodiment, the 3D matrix may comprise Matrigel. Matrigel may comprise laminin, collagen IV, heparan sulfate proteoglycans, entactin/nidogen, TGF-beta, epidermal growth factor, insulin-like growth factor, fibroblast growth factor, tissue plasminogen activator, or combinations thereof.

The 3D matrices may comprise (consist essentially of, or consist of) a naturally derived biopolymer matrix, a synthetic ECM analogue matrix, a hydrogel, a polyethylene glycol (PEG) hydrogel, an RGD functionalized PEG hydrogel, a polyacrylate hydrogel, a hydrogel having a cross-linked hydrophilic polymer functionalized with an RGD-containing peptide, etc.

Hydrogels include naturally derived hydrogels and synthetic hydrogels. Naturally derived hydrogels and synthetic hydrogels may be mixed to form hybrid hydrogels.

Naturally derived hydrogels may include, but are not limited to, Matrigel. Naturally derived hydrogels may be derived from decellularized tissue extracts. Extracellular matrix may be collected from a specific tissue and may be used as or combined with a hydrogel material to be used to support cells of that tissue type. See, e.g., Skardal et al., Tissue Specific Synthetic ECM Hydrogels for 3-D in vitro Maintenance of Hepatocyte Function, Biomaterials 33 (18): 4565-75 (2012). Chitosan hydrogel is an example of a naturally derived hydrogel that is degradable and supportive for several different cell types. See, e.g., Moura et al., In Situ Forming Chitosan Hydrogels Prepared via Ionic/Covalent Co-Cross-Linking, Biomacromolecules 12 (9): 3275-84 (2011). Hyaluronic acid hydrogels may also be used. See, e.g., Skardal et al., A hydrogel bioink toolkit for mimicking native tissue biochemical and mechanical properties in bioprinted tissue constructs, Acta Biomater. 25: 24-34 (2015).

Synthetic hydrogels may be produced from a variety of materials (e.g., polyethylene glycol). By combining natural components, such as extracellular matrix molecules (e.g., extracellular matrix proteins), with synthetic hydrogels, hybrid hydrogels can be produced. See, e.g., Salinas et al., Chondrogenic Differentiation Potential of Human Mesenchymal Stem Cells Photoencapsulated within Poly(Ethylene Glycol)-Arginine-Glycine-Aspartic Acid-Serine Thiol-Methacrylate Mixed-Mode Networks, Tissue Engineering 13 (5): 1025-34 (2007).

A hydrogel may be a matrix comprising a network of hydrophilic polymer chains. A biofunctional hydrogel may be a hydrogel that contains bio-adhesive (or bioactive) molecules, and/or cell signaling molecules that interact with living cells to promote cell viability and a desired cellular phenotype. Biofunctional hydrogels may also be referred to as bioactive. Examples of bio-adhesive molecules include, but are not limited to, fibronectin, vitronectin, bone sialoprotein, laminin, collagen and elastin. Examples of bio-adhesive molecules include cell adhesion peptides such as fibronectin-derived RGD. The hydrogels may comprise a hydrophilic polymer crosslinked with a functional molecule, where the functional molecule may comprise an oligopeptide, a small molecule, a protein, an oligo- or polysaccharides, or an oligo- or poly-nucleotides. The functional molecule may be an RGD-containing ligand such as fibronectin or a functional variant thereof, where the functional variant of fibronectin may be a linear, branched or cyclic peptide.

In some embodiments, the hydrophilic polymer may be polyethylene glycol, polyoxazoline, polyaliphatic polyurethanes, polyether polyurethanes, polyester polyurethanes, polyethylene copolymers, polyamides, polyvinyl alcohols, polyethylene oxide, polypropylene oxide, polypropylene glycol, polytetramethylene oxide, polyvinyl pyrrolidone, polyacrylamide, polyhydroxy ethyl acrylate, poly(hydroxyethyl methacrylate), or mixtures or co-polymers thereof.

Hydrogel precursors may be linear PEG molecules, or multi-arm PEG hydrogel precursor molecules, such as those bearing 4-arms or 8-arms. Hydrogel precursors may be PEG hydrogel precursor molecules with molecular weight of 10-40 kDa. U.S. Pat. No. 10,934,529, the disclosure of which is incorporated herein by reference.

In certain embodiments, the 3D matrix may be a biomatrix scaffold. The biomatrix scaffold may comprise collagens, fibronectins, laminins, nidogen/entactin, elastin, proteogylcans, glycosaminoglycans, growth factors, cytokines or combinations thereof. Biomatrix scaffold may be an isolated tissue extract enriched in extracellular matrix, which retains many or most of the collagens and collagen-bound factors found naturally in the biological tissue. Exemplary collagens include all types of collagens, such as Type I through Type XXIX collagens. U.S. Pat. No. 10,246,678, the disclosure of which is incorporated herein by reference.

When culturing the LBO in a 3D matrix to form a BLBO, the LBO may be embedded through a 3D matrix sandwich (e.g., a Matrigel sandwich). This arrangement of the 3D matrix (e.g., Matrigel) and LBOs allows for 3-dimensional growth of LBOs into BLBOs. Embedding of the LBO may comprise (i) solidifying a first amount of the 3D matrix (e.g., Matrigel) to form a lower portion of solidified 3D matrix (e.g., Matrigel), (ii) solidifying a mixture of the at least one LBO and a second amount of the 3D matrix (e.g., Matrigel) on top of the lower portion to form a solidified center portion; and (iii) solidifying a third amount of the 3D matrix (e.g., Matrigel) on the solidified center portion to form a top portion. In one specific example, the arrangement involves a bottom portion of solidified Matrigel, a mixed Matrigel/LBO middle section, and a top portion of solidified Matrigel, thereby resembling a sandwich configuration.

The concentration of the 3D matrix may range from about 30% to about 100%, from about 40% to about 100%, from about 50% to about 100%, at least or about 20%, at least or about 30%, at least or about 40%, at least or about 50%, at least or about 60%, at least or about 70%, at least or about 80%, at least or about 90%, or about 100%.

A stem cell may refer to a totipotent, pluripotent, multipotent, oligopotent or unipotent cell that can undergo self-renewing cell division to give rise to phenotypically and genotypically identical daughter cells for an indefinite time and can ultimately differentiate into at least one final cell type. The term “stem cell” may mean a cell derived from any source of tissue or organ and can replicate as undifferentiated or lineage committed cells and have the potential to differentiate into at least one, preferably multiple, cell lineages.

Examples of stem cells include totipotent, pluripotent, multipotent, oligopotent and unipotent stem cells (e.g., progenitor cells). Examples of pluripotent stem cells include embryonic stem cells, embryonic germ cells, embryonic carcinoma cells, and induced pluripotent stem cells (iPSCs). Non-limiting examples of stem cells include embryonic stem cells, fetal stem cells, and adult (or somatic) stem cells. Stem cells can be obtained commercially, or obtained/isolated directly from patients, or from any other suitable source.

A stem cell may also be undifferentiated or partially differentiated precursor cells, such as embryonic germ cells, mesenchymal stem cells, multipotent adult stem cells, etc.

In one embodiment, the stem cell is a human stem cell.

Pluripotent stem cells (PSCs) may include embryonic stem cells (ESCs) and induced pluripotent stem cells (iPSCs). In certain embodiments, embryonic stem cells or iPSC cells are undifferentiated pluripotent stem cells, expressing OCT4, SOX2, NANOG, and SSEA4.

ESCs may have unlimited self-renewal and multipotent and/or pluripotent differentiation potential, thus possessing the capability of developing into any organ, tissue type or cell type. These cells can be derived from the inner cell mass of the blastocyst, or from the primordial germ cells from a post-implantation embryo (embryonal germ cells or EG cells). Evans et al. (1981) Nature 292:154-156; Matsui et al. (1991) Nature 353:750-2; Thomson et al. (1995) Proc. Natl. Acad. Sci. USA. 92:7844-8; Thomson et al. (1998) Science 282:1145-1147; and Shamblott et al. (1998) Proc. Natl. Acad. Sci. USA 95:13726-31.

“Induced pluripotent stem cells,” commonly abbreviated as iPS cells or iPSCs, refer to a type of pluripotent stem cell artificially prepared from a non-pluripotent cell, for example an adult somatic cell, or terminally differentiated cell, such as a fibroblast, a hematopoietic cell, a myocyte, a neuron, an epidermal cell, or the like, by introducing certain factors, referred to as reprogramming factors. iPSCs may be generated by reprogramming somatic cells to a pluripotent state. In one aspect, the iPSC is derived from a fibroblast cell. For example, patient fibroblast cells can be collected from the skin biopsy and transformed into iPS cells. Dimos J T et al. (2008) Induced pluripotent stem cells generated from patients with ALS can be differentiated into motor neurons. Science 321: 1218-1221; Nature Reviews Neurology 4, 582-583. Luo et al., Generation of induced pluripotent stem cells from skin fibroblasts of a patient with olivopontocerebellar atrophy, Tohoku J. Exp. Med. 2012, 226(2): 151-9.

In certain embodiments, lung progenitor cells may be generated from a patient-specific source (e.g., iPSC cells), which can provide cell-based regenerative treatments for repopulating healthy lung tissue in diseased patient lungs.

In certain embodiments, the iPSC cells may be from a subject having at least one mutation in a lung disease-associated gene, and the iPSC cells have been genetically altered to correct the gene mutation. In one embodiment, the iPSCs may be genetically altered via the CRISPR/Cas system. In one embodiment, the CRISPR/Cas9 system is used to introduce patient mutations into the stem cell.

The present lung progenitor cells, cell population, artificial lung organoid, or pharmaceutical composition may be administered to a subject to treat a pulmonary disorder or injury.

In some embodiments, the present lung progenitor cells may be used to correct lung-related congenital defects caused by genetic mutations. In particular, mutation affecting human lung development can be corrected using genetically normal three-dimensional artificial lung constructs and/or lung organoids produced from the described methods. In some embodiments, the present lung progenitor cells may be used to generate replacement tissue.

In some embodiments, the present lung progenitor cells may be used to generate replacement lung tissue for lung related disorders.

The CRISPR/Cas system may be used to generate or correct lung disease related gene mutations. The genetically corrected or mutated cell line is then developed into lung progenitor cells.

In one embodiment, the pulmonary disorder or injury is an airway lung disease and/or a distal lung disease. In another embodiment, the pulmonary disorder or injury is pulmonary fibrosis. In another embodiment, the pulmonary disorder or injury is a non-malignant lung disease. In yet another embodiment, the pulmonary disorder or injury is an interstitial lung disease (including congenital interstitial lung diseases, etc.). In certain embodiments, the pulmonary disorder or injury may be a congenital surfactant deficiency.

Non-limiting examples of pulmonary disorders or injuries include, cystic fibrosis; emphysema; chronic obstructive pulmonary disease (COPD); interstitial lung diseases including pulmonary fibrosis, idiopathic pulmonary fibrosis (IPF), Hermansky-Pudlak Syndrome (HPS), hypersensitivity pneumonitis, sarcoidosis, asbestosis, autoimmune-mediated interstitial lung disease; pulmonary hypertension; lung cancer; acute lung injury (adult respiratory distress syndrome); respiratory distress syndrome of prematurity, chronic lung disease of prematurity (bronchopulmonary dysplasia); congenital surfactant deficiencies, including surfactant protein B deficiency, surfactant protein C deficiency, ABCA3 deficiency; ciliopathies; congenital diaphragmatic hernia; pulmonary alveolar proteinosis; pulmonary hypoplasia; lung injury, and combinations thereof. The pulmonary disorder or injury may be HPS-associated interstitial pneumonia (HPSIP).

Pulmonary fibrosis is the formation or development of excess fibrous connective tissue (fibrosis) in the lungs, also described as “scarring of the lung.” Pulmonary fibrosis may be a secondary effect of other diseases. Most of these are classified as interstitial lung diseases. Examples include autoimmune disorders, viral infections or other microscopic injuries to the lung. However, pulmonary fibrosis can also appear without any known cause (termed “idiopathic”), and differs from other forms of fibrosis in that it is not responsive to any immune suppressive treatment.

A subset of patients with Hermansky-Pudlak Syndrome (HPS) shows a high incidence of IPF, also called HPS-associated interstitial pneumonia (HPSIP). HPS is an autosomal recessive disorder affecting the biogenesis and trafficking of lysosome related organelles (LROs). All HPS mutations cause bleeding diathesis due to lack of platelet delta granules, albinism due to improper melanosome formation in melanocytes, and in some genotypes pulmonary fibrosis due to abnormal LB trafficking.⁵¹ HPS1, HPS2 and HPS4 mutations cause HPS-associated interstitial pneumonia (HPSIP), a disease very similar to IPF with high penetrance, while other HPS mutations, such as HPS8, almost never cause pulmonary disease. The mutations causing HPS may affect four distinct protein complexes: biogenesis of lysosome-related organelle complex (BLOC1 (HPS7, HPS 8, HPS9), BLOC2 (HPS3, HPS5, HPS6), BLOC3 (HPS1, HPS4) and AP3 (HPS2)).

Alternative approaches to treat diseased lung and airways include the use of tissues reconstituted within decellularized lung matrices. The present lung progenitors may be used to seed a decellularized lung matrix.

The present lung progenitor cells and organoids may be used for studying human lung development, modeling lung diseases (e.g., such as RSV infection and fibrosis), testing therapeutic agents, screening drugs, and regenerative medicine.

In some embodiments, the present lung progenitor cells may be used to identify the molecular basis of normal human lung development.

In some embodiments, the present lung progenitor cells may be used to identify the molecular basis of congenital defects affecting human lung development.

Diseases that can be studied using the present lung progenitor cells or lung organoids include genetic diseases, metabolic diseases, pathogenic diseases, inflammatory diseases, etc.

The present lung progenitor cells or lung organoids can be used for culturing of a pathogen and thus can be used as ex vivo infection models. Examples of pathogens that may be cultured using the present lung progenitor cells or lung organoids include viruses, bacteria, prions or fungi that cause disease in its animal host. Thus, the present lung progenitor cells or lung organoids can be used as a disease model that represents an infected state. For example, the present lung progenitor cells may be used to model the morphological features of respiratory syncytial virus (RSV) infection in the human lung (e.g., the distal lung). For example, lung progenitor cells may be generated from RUES2 cells and then infected with RSV.

Lung progenitor cells may be generated from mutated stem cells to study lung diseases including fibrosis, surfactant secretion disease, or cystic fibrosis. The mutated stem cells may have mutations in HPS1, HPS2, HPS3, HPS5, HPS8, ABCA3, and/or telomerase.

To recapitulate fibrosis in vitro, lung progenitor cells may be generated from RUES2 cells carrying a deletion of the HPS1 gene (e.g., engineered using CRISPR/Cas9) which predisposes the cells with high penetrance to IPF.

In certain embodiments, when patient-specific iPSC lines are generated from cystic fibrosis patients, either before or after gene editing to correct the CFTR genetic lesion responsible for the disease, the present lung progenitor cells or lung organoids allow precise interrogation of mutant versus corrected CFTR function through forskolin-induced epithelial sphere swelling assays.

In certain embodiments, iPSCs derived from patients harboring a lung disease related genetic mutation can be corrected, in vitro, using the CRISPR/Cas system to produce a genetically corrected cell line. Production of lung progenitor cells using cells that have been genetically altered for correcting a genetic defect provides a method of testing such genetic alterations for their capacity to correct the disease phenotype.

The term “lung-disease related mutation” as used herein relates to a gene mutation or polymorphism known to cause a lung disease phenotype. For example, certain lung diseases are caused by gene mutations in one or more of the following, non-exhaustive list of genes: HPS1 (gene ID 3257), HPS2 (gene ID 7031; TFF1), HPS4 (gene ID 89781), TERT (e.g., hTERT, gene ID 7015), TERC (e.g., hTERC; gene ID 7012), dyskerin, CFTR (gene ID 1080), DKC1 (gene ID 1736), SFPTB (gene ID 6439), SFTPC (gene ID 6440), SFTPA1 (gene ID 653509), SFTPA2 (gene ID 729238), MUC5B (gene ID 727897), SHH (gene ID 6469), PTCH (e.g., PTCH1; gene ID 5727), SMO (gene ID 6608), ABCA3 (gene ID 21), PARN, RTEL1, and KIF15.

HPS1 (Online Mendelian Inheritance in Man (OMIM) #604982) is part of BLOC3. Mutation in HPS1 is the most penetrant for PF (currently 80%). Multiple mutations have been described, all of which eliminate BLOC3. For example, a frame shift hot-spot at codons 321-322 may be used to elicit fibrosis in vitro.

HPS2 (OMIM #608233) mutation destabilizes the AP3 complex, and also predisposes to fibrosis. As multiple deletions and frame shifts in AP3B1 cause nonsense-mediated mRNA decay, thus deleting the entire protein and the AP3 complex. A deletion may be introduced in the 5′ region.

HPS8 (OMIM #614077): Mutation in BLOC1S3, part of the BLOC1 complex, causes a form of HPS that is not associated with IPF and serves as a control. The initial mutation described is a 1 bp frameshift deletion that theoretically gives rise to abnormal 244 aa protein as nonsense-mediated mRNA decays was not observed. Another human mutation however did show nonsense-mediated mRNA decay, with mRNA undetectable.147 Deletion of the gene by targeting the 5′ region for frameshift mutation may result in organoids appearing to develop dilated branch tips, which might be suggestive of abnormal surfactant secretion. All HPS genes play a role in the biogenesis of lysosome-related organelles, including lamellar bodies of type II alveolar epithelial cells, and HPS8 may have a surfactant secretion phenotype in vitro.

Telomerase (OMIM #614742): Because IPF is the most common clinical manifestation of mutation in telomerase genes, introduction of telomerase mutations into hESCs may be used to examine the effect of telomeropathy on ATII cell function. Mutations in TERT (e.g., hTERT) and/or TERC (e.g., hTERC) may be introduced. Heterozygous or double heterozygous indels in the N-terminal region of hTERC, or TERC-deleted cells may be used.

HPS5 (OMIM #607521) encodes a protein of the BLOC2 complex. HPS5 is not associated with interstitial lung disease and may serve as a control. The only mutation known in humans is a homozygous 4-bp deletion (AGTT) at codons leu675 to val676. The mutation resulted in a frameshift with truncation of the nonsense polypeptide at codon 682, causing loss of 40% of the protein at the C terminus.

HPS3 (OMIM #060118) is part of the BLOC2 complex. HPS3 is not associated with interstitial lung disease, and may serve as a control. Deletion in the 5′ region of the HPS gene may be used.

LYST (OMIM #606897): Multiple frame shift mutations have been described that give rise to severe childhood onset Chediak-Higashi syndrome (CHS) with confirmed giant granules in white blood cells and melanocytes. An indel at Lys633/Lys634, which results in a premature stop a codon 638, may be used.

SFTPC (OMIM #178620): A heterozygous T->A transversion in nucleotide 128 of exon 5 may be introduced, using a guide RNAs and a homologous single stranded 80 bp DNA segment containing the point mutation. This heterozygous mutation can cause highly penetrant IPF.

Cystic fibrosis is associated with gene mutations in the cystic fibrosis transmembrane conductance regulator (CFTR) and polymorphisms associated sodium channel epithelial 1 alpha (SCNN1A) genes, and such mutations/polymorphisms are highly variable. With reference to the expressed proteins of such genes, the mutations include F508 in the a CFTR protein, G551 in a CFTR protein, G542 in a CFTR protein, N1303 in a CFTR protein, R117 in a CFTR protein, W1282 in a CFTR protein, R553 in a CFTR protein, c.3849+10 kb in a CFTR protein, c.2657+5 in a CFTR protein, c.3140-26 in a CFTR protein, and V114 in a SCNN1A protein. The mutations also include SFTPB121ins2.

Cells harboring mutated genes including, but not limited to, those described above, can be subjected to a CRISPR/Cas system. For example, the cells may be subjected to the CRISPR/Cas induced genetic correction at a stage of growth and expansion such at a pluripotent stage. These cells would then be developed into lung progenitor cells as described herein.

The present lung progenitor cells or lung organoids may be used for agent or vaccine screening (e.g., screening for efficacy, toxicity, or other metabolic or physiological activity) or for treatment of (including resistance to treatment of) lung infection, diseases, and injuries. In particular, the present methods, cells, organoids, compositions, and kits can be used in assays, e.g., high-throughput assays, e.g., for the discovery of agents to treat lung disorders and injuries.

The present lung progenitor cells or lung organoids may be used to study drug delivery. In some embodiments, the present lung progenitor cells may be used to screen drugs for lung tissue uptake and mechanisms of transport. For example, this can be done in a high throughput manner to screen for the most readily absorbed drugs, and can augment Phase 1 clinical trials that are done to study drug lung tissue uptake and lung tissue toxicity. This includes pericellular and intracellular transport mechanisms of small molecules, peptides, metabolites, salts.

The present lung progenitor cells may be used to screen for a test agent. The present screening method may comprise contacting a test agent or a library of agents with the present lung progenitor cells.

In some embodiments, the organoids can be used in vaccine development and/or production. Methods of determining whether a test agent has immunological activity may include testing for immunoglobulin generation, chemokine generation and cytokine generation by the cells.

For acute treatment testing, an agent or vaccine may be applied, e.g., once for several hours. For chronic treatment testing, an agent or vaccine may be applied, e.g., for days to one week. Such testing may be carried out by providing a lung organoid under suitable conditions (e.g., in a culture medium with oxygenation); applying an agent to be tested (e.g., a drug candidate) to the lung organoid (e.g., by topical or vapor application); and then detecting a physiological response (e.g., damage, scar tissue formation, infection, cell proliferation, burn, cell death, marker release such as histamine release, cytokine release, changes in gene expression, etc.), the presence of such a physiological response indicating said agent or vaccine has therapeutic efficacy, toxicity, or other metabolic or physiological activity if inhaled or otherwise delivered into the lung of a mammalian subject. A control sample of the lung organoid may be maintained under like conditions, to which a control agent (e.g., physiological saline, compound vehicle or carrier) may be applied, so that a comparative result is achieved.

In certain embodiments, the present lung progenitor cells may be used in methods for screening for a test agent that can treat certain condition(s). For example, agents may be screened for preventing or reducing the formation of collagen using the present lung progenitor cells. Agents may be screened for preventing or reducing fibrosis using the present lung progenitor cells. Agents may be screened for preventing or reducing the formation of fibronectin and/or any other extracellular matrix protein, as well as mesenchymal cells (fibroblast, lipofibroblast, myofibroblasts, etc.) using the present lung progenitor cells. Agents may be screened for increasing or decreasing surfactant production using the present lung progenitor cells.

Agents may be screened for treating fibrosis using lung progenitor cells having mutations in one or more genes that are known to cause fibrosis (e.g., HPS1, HPS2 SFTPC and TERC). Cell lines with mutations in HPS and/or LYST may be used as controls, because these mutations affect lysosome-related organelles but are not associated with clinical fibrosis.

Examples of the agents include a small molecule (e.g., a small organic molecule), a protein, a peptide, an antibody or fragments thereof, a nonpeptidic compound, a synthesis compound, a fermentation product, a cell extract, a plant extract, an animal tissue extract, a nucleic acid (e.g., DNA, RNA), a cell culture supernatant, a plasma, or the like. In other embodiments, types of agents include, but are not limited to, peptide analogs including peptides comprising non-naturally occurring amino acids, e.g., D-amino acids, phosphorous analogs of amino acids, such as α-amino phosphoric acids, or amino acids having non-peptide linkages, nucleic acid analogs such as phosphorothioates and PNAs, hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose.

The present disclosure also provides a kit comprising the present lung progenitor cells, cell population, artificial lung organoid, or pharmaceutical composition. The kit can include a package insert including information concerning cell growth and maintenance, as well as buffers and/or growth factors in the kit.

The present kit may further include containers for suitable administration and a package insert including information concerning the lung progenitor cells, cell population, artificial lung organoid, or pharmaceutical compositions, and dosage forms in the kit. Generally, such information aids researchers, scientists, patients and physicians in using the enclosed lung progenitor cells, cell population, artificial lung organoid, or pharmaceutical compositions effectively and safely. For example, the following information may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

As used herein, definitive endoderm (DE) is one of the three germ layers arising after gastrulation that give rise to the intestinal tract, liver, pancreas, stomach and all other organs derived from the AFE, as listed above. DE expresses the markers: FOXA2, FOXA1, cKIT, CXCR4, and EPCAM.

As used herein, “anterior foregut endoderm” (AFE) refers to endoderm that is anterior to the endoderm that gives rise to the most proximal derivatives of the endoderm or primitive gut tube. Anterior foregut endoderm may include, for example, pharyngeal endoderm or lung endoderm and other, more highly differentiated populations of endodermal cells. As embryonic tissues express characteristic sets of molecular markers, the various cell types encompassed by the term “anterior foregut endoderm” may exhibit different expression patterns of molecular markers. Anterior foregut endoderm can give rise to various tissues, e.g., tonsils, tympanic membrane, thyroid, parathyroid glands, thymus, trachea, esophagus, stomach, lung and larynx/pharynx. Anterior foregut endoderm expresses FOXA2, FOXA1, SOX2 and EPCAM and is negative for the distal endoderm marker CDX2.

An organoid may refer to an artificial, in vitro three-dimensional construct created to mimic or resemble the functionality and/or histological structure of an organ or portion thereof. An organoid may refer to a 3-dimensional growth of mammalian cells in culture that retains characteristics of the tissue in vivo, e.g., prolonged tissue expansion with proliferation, multilineage differentiation, recapitulation of cellular and tissue ultrastructure, etc.

A lung bud organoid (LBO) may contain lung epithelial cells (expressing FOXA2, FOXA1, NKX2.1 and EPCAM) and/or mesenchymal progenitors (expressing PDGFRa, CD90, TBX4, and HOXA5). Lung bud organoids may generate branching colonies after embedding in a 3D matrix (e.g., Matrigel). LBOs may be spheroids when generated from anterior foregut endoderm cells in suspension cultures in vitro. LBOs may form between day 15 to day 25 and may include folding structures.

The term “branched LBO” (BLBO) as used herein refers to LBOs that possess structures relating to branching morphogenesis. As the BLBOs further develop they begin to show dilated tips which have the morphology of fetal alveolar structures.

As used herein, a “therapeutically effective” amount is an amount of an agent effective to treat, ameliorate or lessen a symptom or cause of a given pathological condition in a subject suffering therefrom to which the agent is to be administered.

As used herein, a “prophylactically effective” amount is an amount of an agent effective to prevent or to delay the onset of a given pathological condition in a subject to which the substance is to be administered. A prophylactically effective amount refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

“Treating” or “treatment” of a state, disorder or condition includes: (1) preventing or delaying the appearance of clinical symptoms of the state, disorder, or condition developing in a person who may be afflicted with or predisposed to the state, disorder or condition but does not yet experience or display clinical symptoms of the state, disorder or condition; or (2) inhibiting the state, disorder or condition, i.e., arresting, reducing or delaying the development of the disease or a relapse thereof (in case of maintenance treatment) or at least one clinical symptom, sign, or test, thereof; or (3) relieving the disease, i.e., causing regression of the state, disorder or condition or at least one of its clinical or sub-clinical symptoms or signs.

The term “subject” includes any animal, preferably a mammal (e.g., rat, mouse, dog, cat, rabbit) and more preferably a human. Subjects, which may be treated according to the present disclosure, include all animals which may benefit from the present invention. Such subjects include mammals. “Patient” or “subject” refers to mammals and includes human and veterinary subjects. Certain veterinary subjects may include avian species.

“Mammalian” and “mammals” as used herein refers to both human subjects (and cell sources) and non-human subjects (and cell sources or types), such as dogs, cats, rats, mice, rabbits, monkeys, etc. (e.g., for veterinary purposes). Mammals include humans (infants, children, adolescents and/or adults), and animals such as dogs and cats, farm animals such as cows, pigs, sheep, horses, goats and the like, and laboratory animals (e.g., rats, mice, guinea pigs, and the like).

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. The term “pharmaceutically acceptable” may mean approved by a regulatory agency of the Federal or a state government or listed in the U.S. Pharmacopoeia or other generally recognized pharmacopeias for use in animals, and more particularly in humans. Pharmaceutically acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

The term “allogeneic” refers to any material derived from a different animal of the same species as the individual to whom the material is introduced. Two or more individuals are said to be allogeneic to one another.

The term “autologous” refers to any material derived from the same individual to whom it is later to be re-introduced into the same individual.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%, 10% or 5%.

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, NY; Sambrook and Russell (2001) Molecular Cloning, 3rd ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

The following examples of specific aspects for carrying out the present disclosure are offered for illustrative purposes only, and are not intended to limit the scope of the present disclosure in any way.

### Example 1

We have generated expandable, clonal spheres, called “transitional lung organoids” (TLOs), from human pluripotent stem cells. TLOs contain mainly type 0 alveolar epithelial (AT0) cells, terminal respiratory bronchiole stem cells (TRB-SCs) and distal basal cells (BCs).³ Velocity analysis of single cell RNAseq data suggests that distal BCs are the most undifferentiated progenitors in the TLOs and give rise to AT0 cells, which are likely precursors of AT2 cells. Introduction of mutations involved in idiopathic pulmonary fibrosis (IPF) induced profound changes in gene expression, differentiation capacity and apoptosis, associated with defective ER stress and integrated stress responses (ISR). These findings indicate not only mature AT2 cells, but also their precursors, are functionally affected in IPF and show a potential key role for the ISR. We furthermore show how these can be used to model mechanisms involved in IPF. TLOs may be used as a model for studies in human lung regeneration, disease modeling, and drug target identification and validation.

### 1. Generation of TLOs

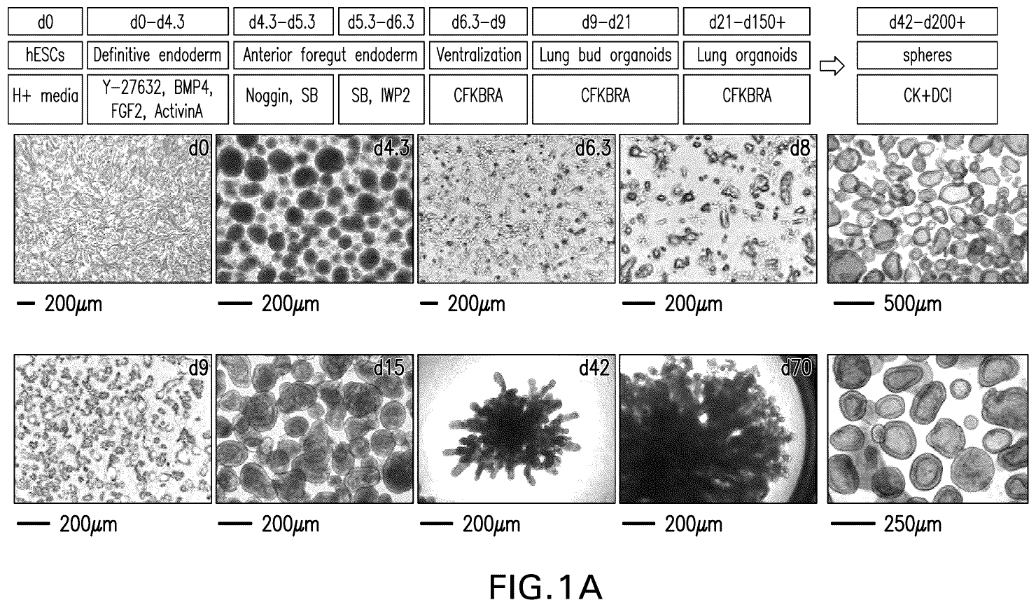

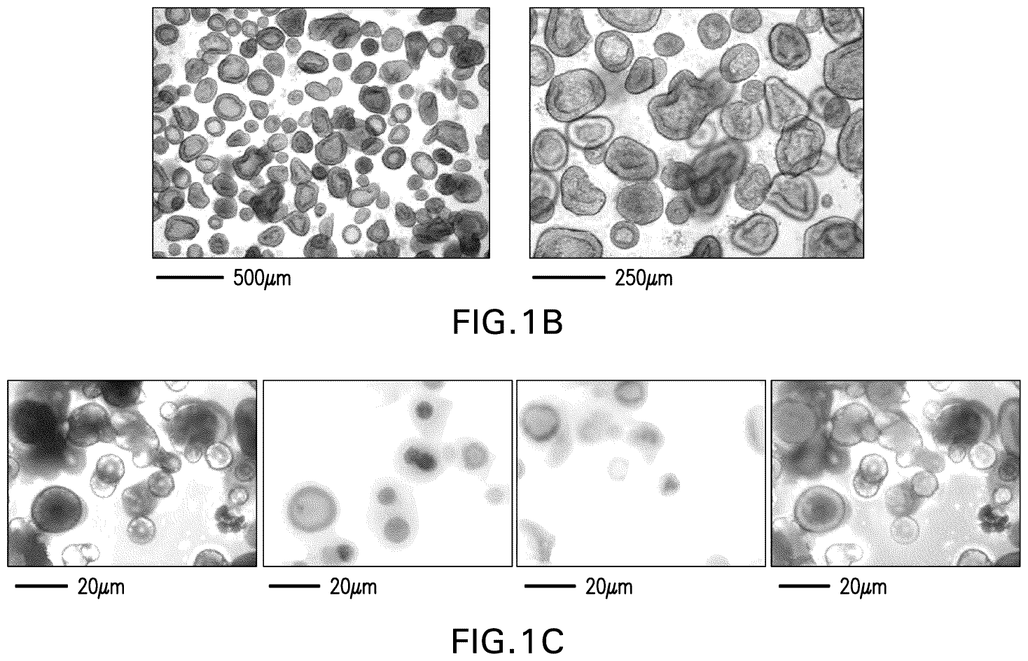

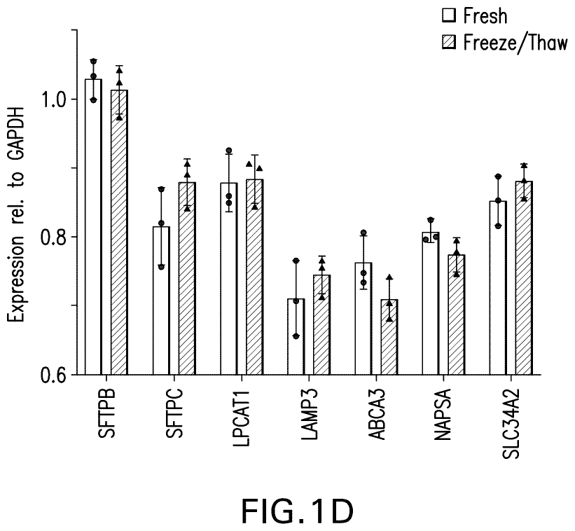

We previously reported the generation of fetal stage, branching distal lung organoids from hPSCs.²⁸ After dissociation of ESC or iPSC-derived organoids and culture as single cells in Matrigel with CK-DCI (GSK3 inhibitor CHIR, keratinocyte growth factor (KGF) which is also called FGF7, dexamethasone, 3′,5′-cyclic adenosine monophosphate (cAMP), and 3-isobutyl-1-methylxanthine (IBMX)), hollow spheres developed within 2 weeks (FIGS. 1a, 1b), which we call “transitional lung organoids” (TLOs). TLOs were clonal as mixing single cells from organoids expressing GFP or mScarlet yielded only spheres expressing either fluorescent reporter (FIG. 1c). TLOs can be serially passaged about every 3 weeks for up to at least 7 passages with an average expansion of about 8-fold between each passage. Over this time, TLOs were phenotypically stable. Furthermore, they can be cryopreserved and thawed while retaining their expression profile (FIG. 1d).

### 2. Characterization of TLOs

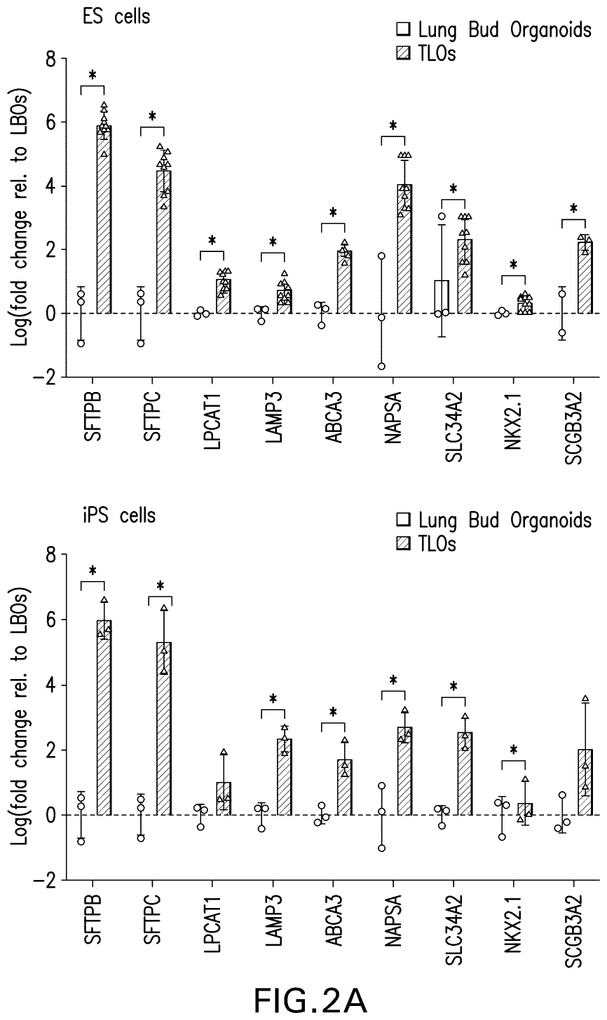

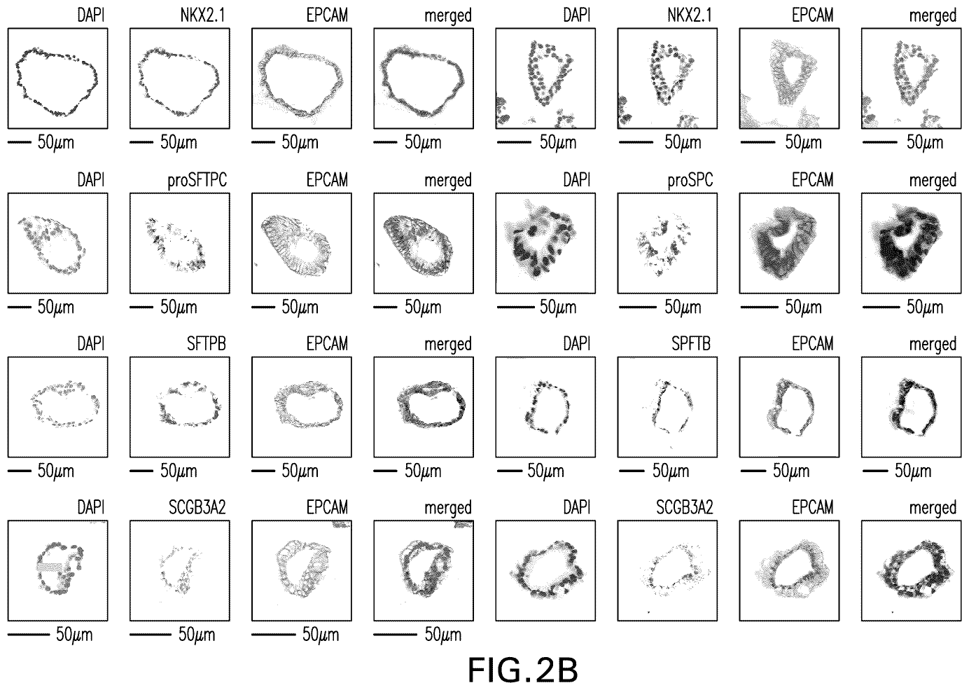

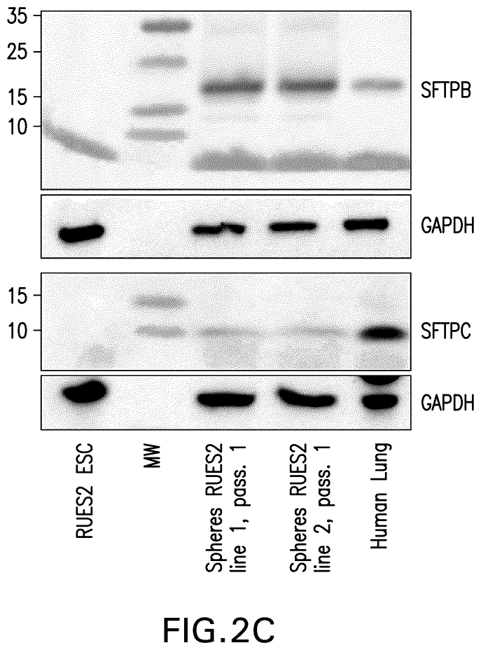

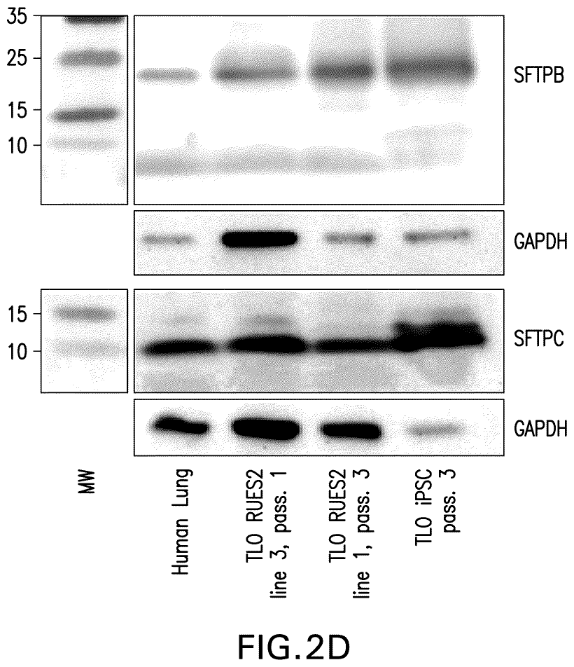

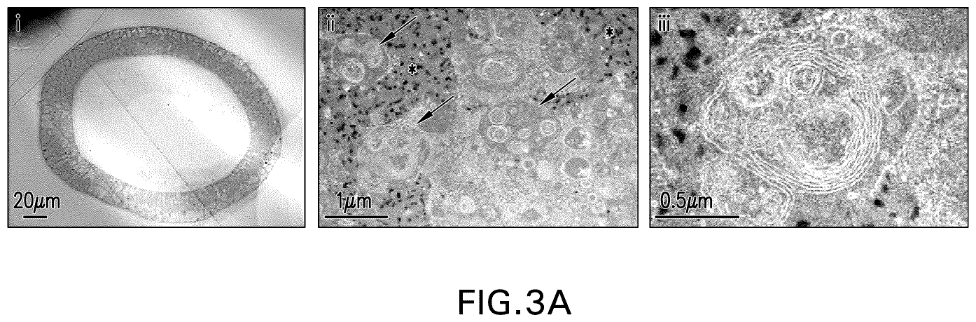

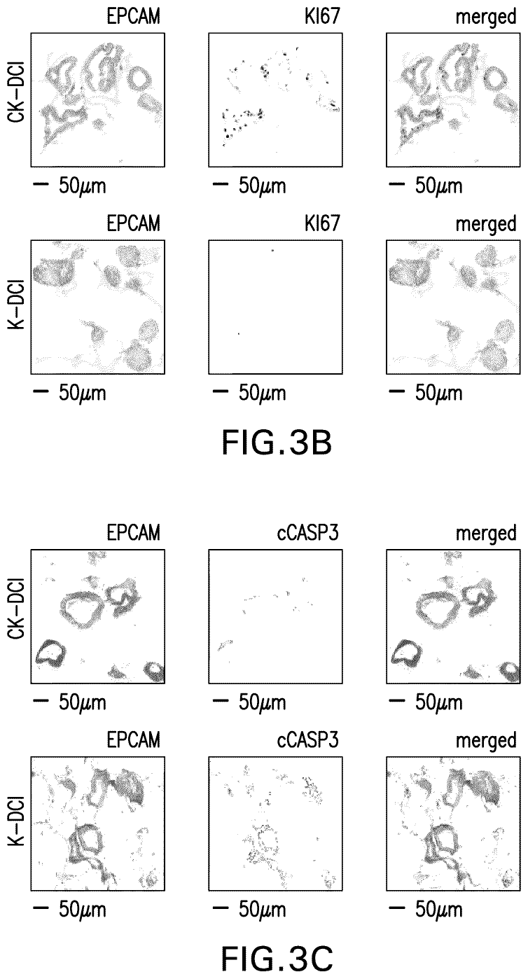

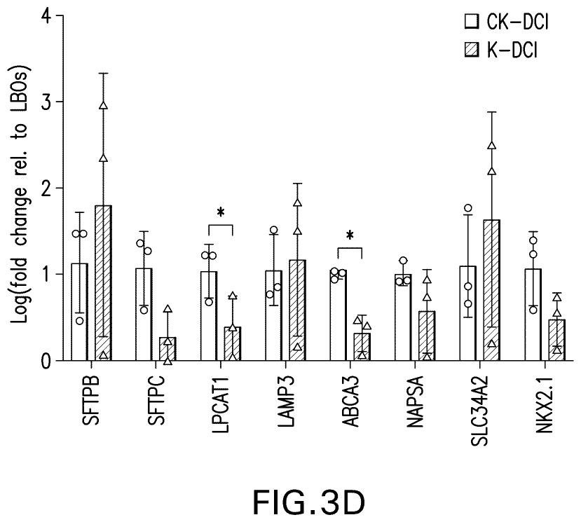

TLOs highly expressed mRNAs encoding the AT2 markers, SFTPC, SFTPB, ABCA3, LPCAT, NAPSA, SLC234A2, and LAMP3 (FIG. 2a) in both RUES ESCs and iPSC-derived TLOs. At variance with previously reported alveolospheres,^{25,26} however, strong induction of SCGB3A2 mRNA was observed (FIG. 2a). Confocal microscopy revealed uniform expression of EPCAM, NKX2.1 and SFTPB with a subset of cells in each TLO also expressing pro-SFTPC and SCGB3A2 (FIG. 2b). Western Blot showed presence of fully processed SFTPC and SFTPB (FIGS. 2c, 2d). Transmission electron microscopy revealed apical presence of lamellar bodies (LBs) in most cells, a feature of AT2 cells,²⁹ (FIG. 3a), consistent with the SFTPB staining pattern (FIG. 2b). However, these had low electron density and were often organized as multivesicular bodies (FIG. 3a, arrows), while the cytoplasm contained glycogen (FIG. 3a stars), indicative of immaturity.^{30,31}

As removing WNT signaling has been reported to induce AT2 maturation in alveolospheres,^{26,27} we withdrew CHIR. CHIR withdrawal, however, led to disintegration, proliferation arrest (FIG. 3b), apoptosis (FIG. 3c) and loss of AT2 markers (FIG. 3c), indicating that GSK3 inhibition is required for TLO maintenance and propagation and that these cells are distinct from AT2 cells.

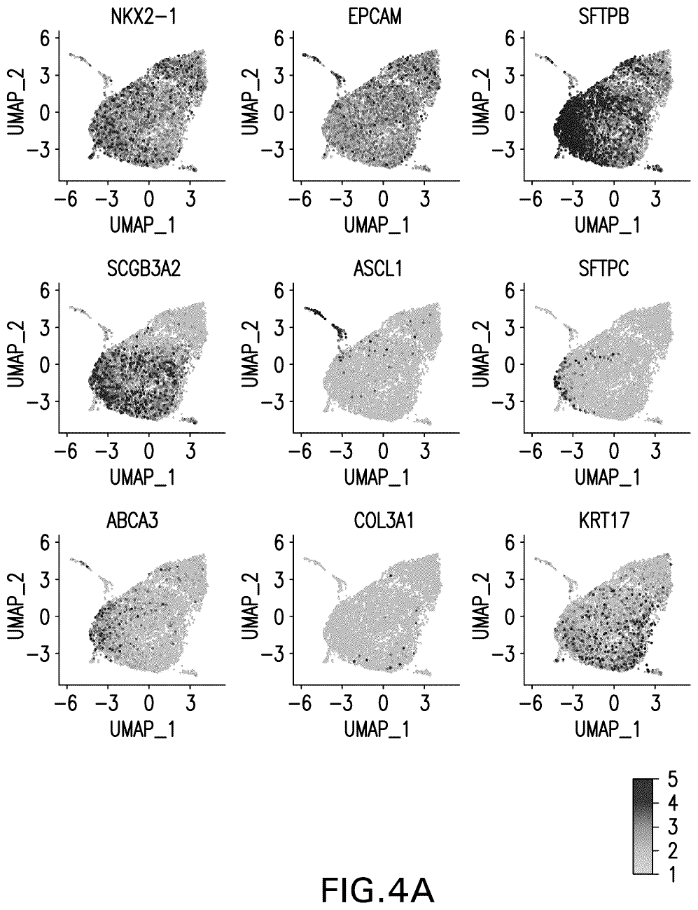

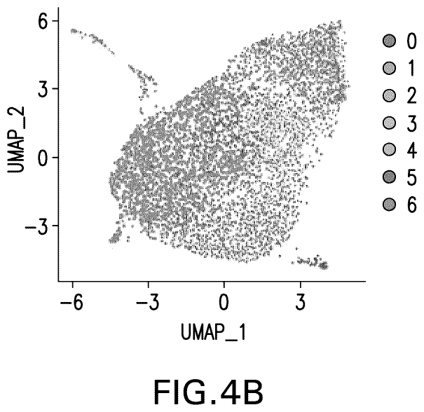

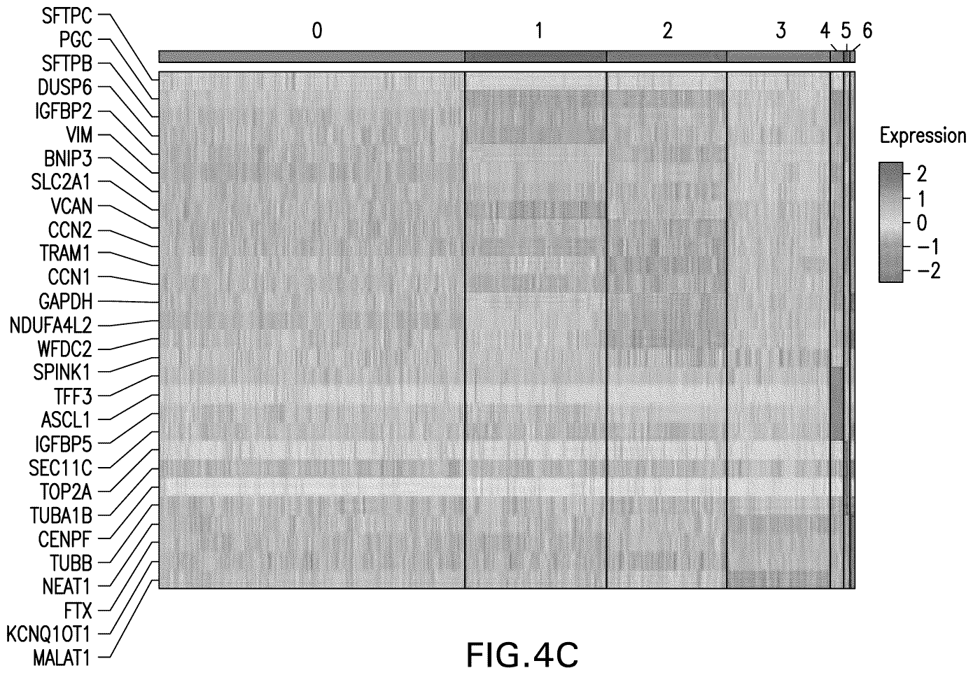

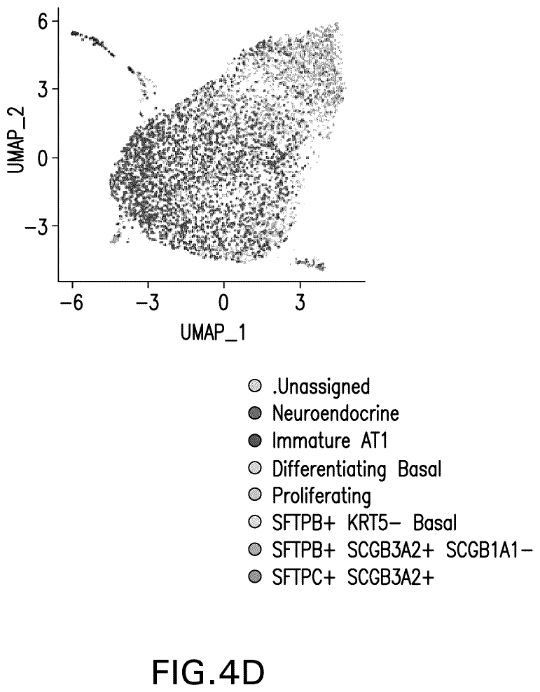

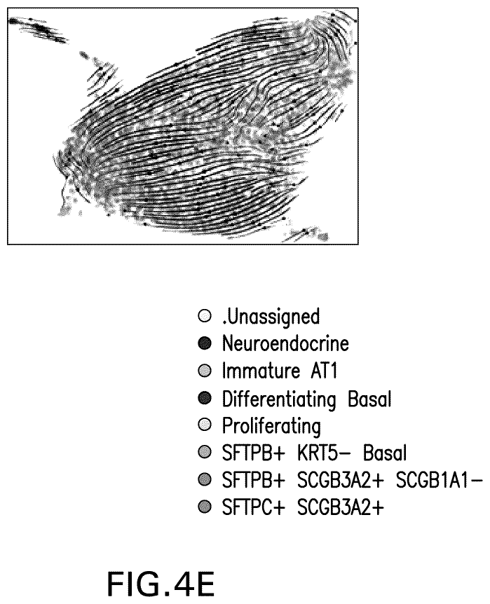

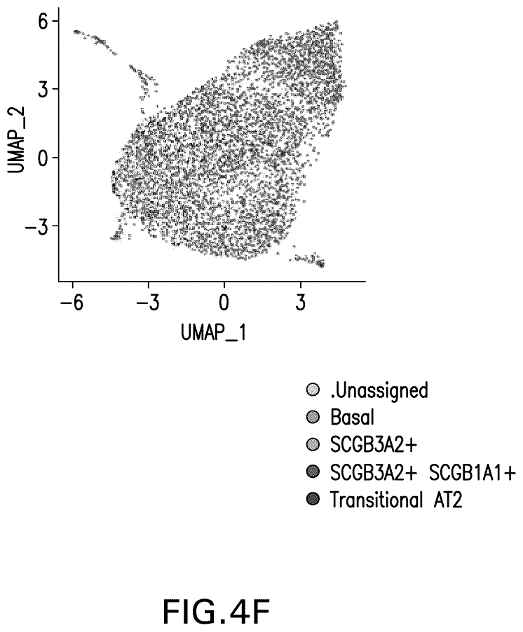

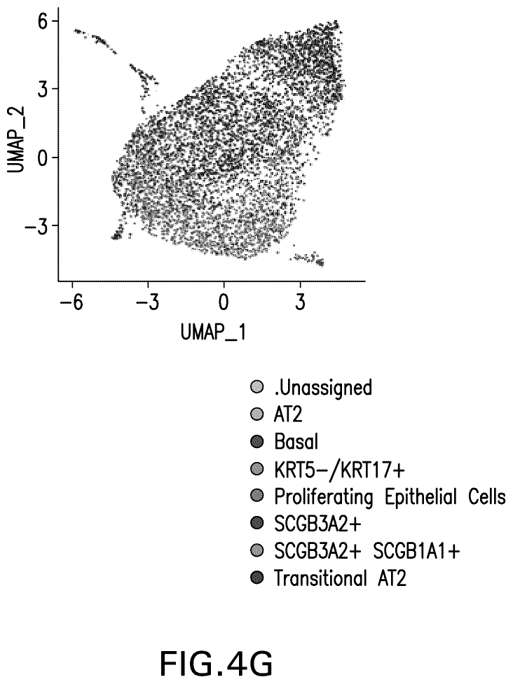

ScRNAseq confirmed that almost all cells expressed SFTPB, NKX2.1 and EPCAM and identified a distinct, large population of SCGB3A2⁺ cells that expressed the most SFTPB (FIG. 4a). Markers of mature airway cells were absent, and, in contrast to previously reported alveolospheres,^{26,27} contamination with gastric or intestinal cells was not detected (not shown). Clustering revealed 4 closely related clusters (0-3), and 3 small but more distinct clusters that each made up ˜1% of the cells (4-6) (FIG. 4b, heatmap FIG. 4c). Cluster 0 contained cells expressing AT2 markers (SFTPC, ABCA3, NAPSA) and the highest levels of SFTPB, while clusters 0, 2 and 3 expressed SCGB3A2 (FIG. 4a). Cluster 1, at the other extreme, expressed less SFTPB but did not express SCGB3A2 or AT2 markers (FIG. 4a). Cell type annotation using machine learning trained on the data of Murthy et al.³ matched cluster 0 with AT0 cells (SFTPB⁺SCGB3A2⁺SFTPC⁺) and TRB-SCs (SFTPB⁺SCGB3A2⁺SFTPC⁻). Cluster 1 corresponded to SFTPB⁺KRT5⁻P63⁻SCGB3A2⁻ distal BCs, primarily characterized by elevated expression of IGFBP2 (FIG. 4d), a marker of airway and in particular of basal cells according to LungMap.³² Clusters 2 and 3 were classified as a mixture of ‘differentiating BCs’ and rare ‘immature AT1 cells’, both likely representing differentiation intermediates (FIG. 4d). No matches with any other lung populations were found. Trajectory analysis using scVelo²⁴ confirmed the notion that clusters 2 and 3 were intermediate between clusters 0 and 1, and showed differentiation pathways originating from distal BCs (cluster 1) and terminating in AT0 cells in cluster 0 (FIG. 4f). Given the clonal nature of the TLOs, these findings suggest a lineage from distal BCs over TRB-SCs to AT0 cells that was not recognized in recent scRNAseq studies of human lung.^{1-3,22,32} Cluster 4 are rare ASCL1⁺ neuroendocrine cells, some of which, quite unusually, also expressed SFTPB and SCGB3A2 (FIG. 4a,c) and may represent precursors. The small cluster 5 contained proliferating cells that could not be further identified, while the equally small cluster 6 is distinguished by four lncRNAs (FIG. 4c), and may also be a regenerative population.

Cell identity assignment based on the data of Haberman et al.,¹ also showed SCGB3A2⁺ cells and transitional SCGB3A2⁺ AT2 cells mainly in cluster 0, and classified the remainder as BCs (they did not describe specific BC subsets) and some rare SCGB3A2⁺SCGB1A1⁺ cells (likely closely related to pre-TRB-SCs from Murthy et al.³) (FIG. 4f). Both training sets for cell identity assignment thus yielded consistent results. Haberman et al.¹ and Adams et al.³³ identified KRT5⁻KRT17⁺ ‘aberrant’ basaloid cells that express mesenchymal markers and are nearly exclusive to lungs of IPF patients. Including the IPF data of Haberman et al. in the training set revealed that cluster 2 corresponded to KRT5⁻KRT17⁺ basaloid cells, which were also found in cluster 0 (FIG. 4g). The distribution of KRT17⁺ cells, some of which expressed the mesenchymal marker, COL3A1, was consistent with this finding (FIG. 4a). It is interesting to note that scVelo analysis suggests that cells in cluster 2 may also be precursors of AT0 cells (FIG. 4e).

The TLOs described here contain almost entirely a spectrum of potential distal lung progenitor cells,¹⁻³ and include KRT5⁻KRT17⁺ basaloid cells mainly found in IPF.^{1,33} TLOs therefore include most cells involved in normal and pathological human distal lung regeneration identified thus far. Despite similar culture conditions, TLOs differ from these previously reported alveolospheres, which, in contrast to TLOs, undergo AT2 maturation after intermittent CHIR withdrawal,^{26,27} require reporter lines or enrichment steps for their generation, and contain gastric-committed cells by scRNAseq.^{25-27,34} Most importantly, TLOs differ from alveolospheres by the abundant presence of SCGB3A2⁺SFPTB⁺ cells that were assigned as TRB-SCs based on the data of Murthy et al.³

As these populations are absent in rodent models, TLOs will be an important resource to gain deeper insight into mechanism involved in normal and abnormal human lung regeneration, and may have applications in regenerative approaches for lung diseases.

### 3. Differentiation Capacity of TLOs

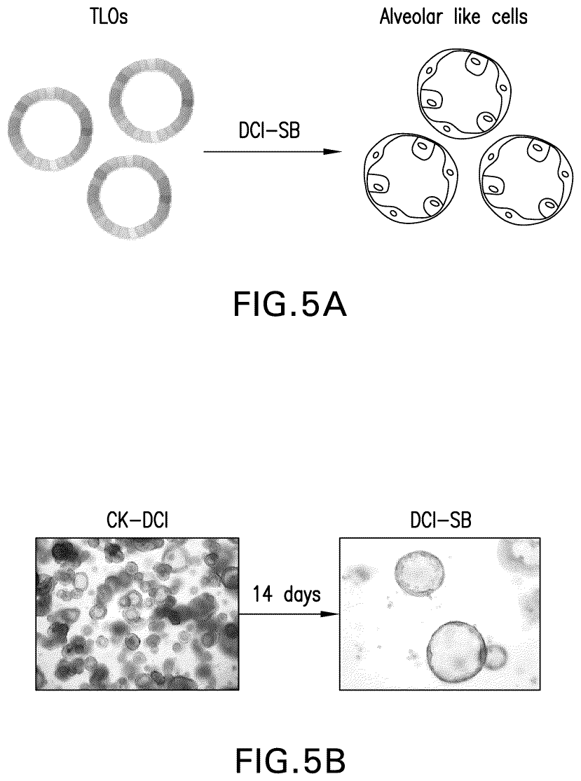

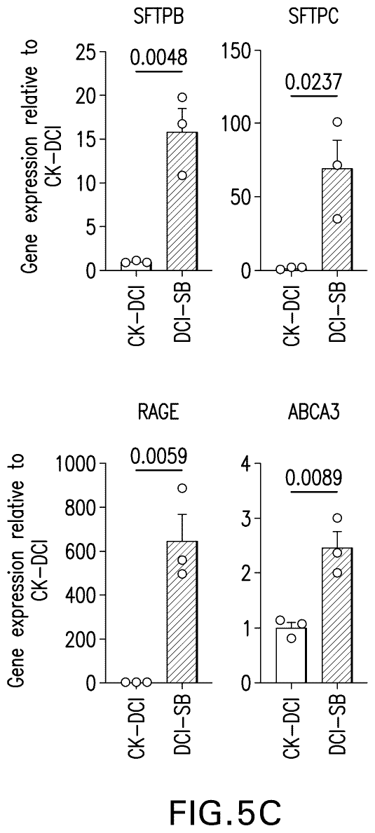

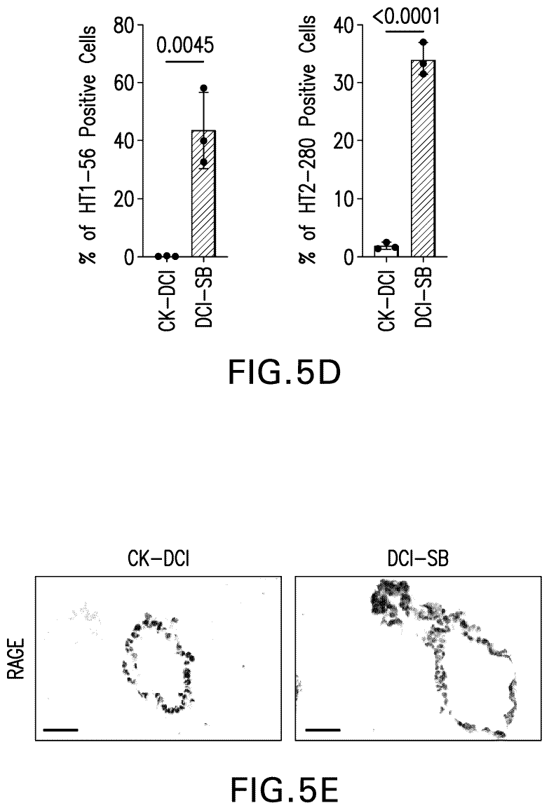

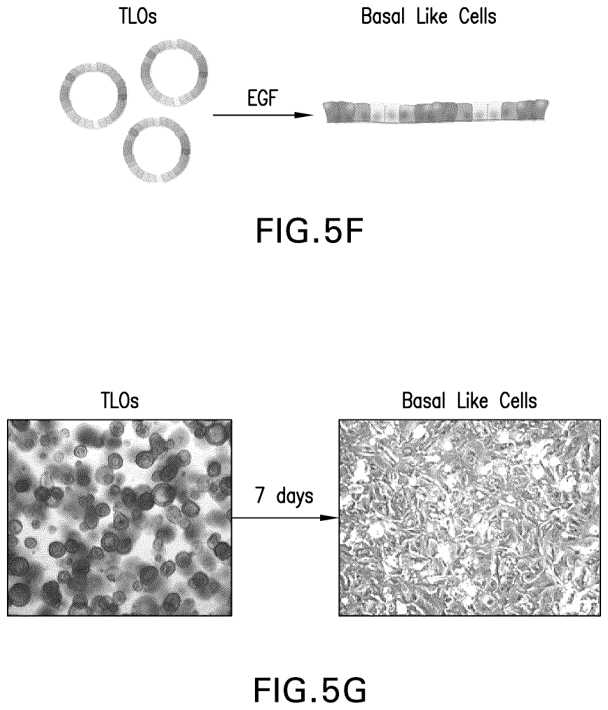

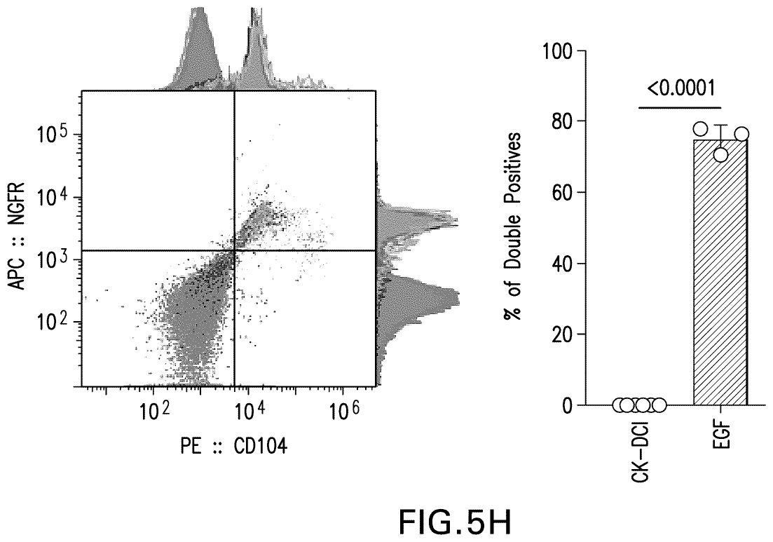

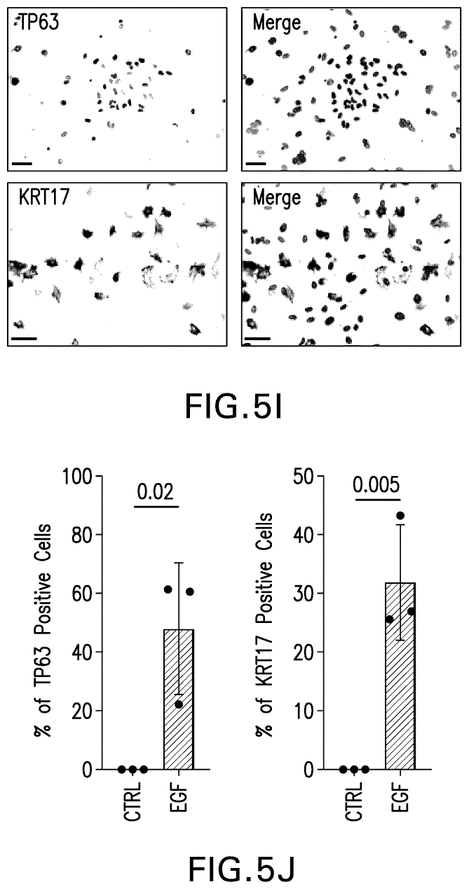

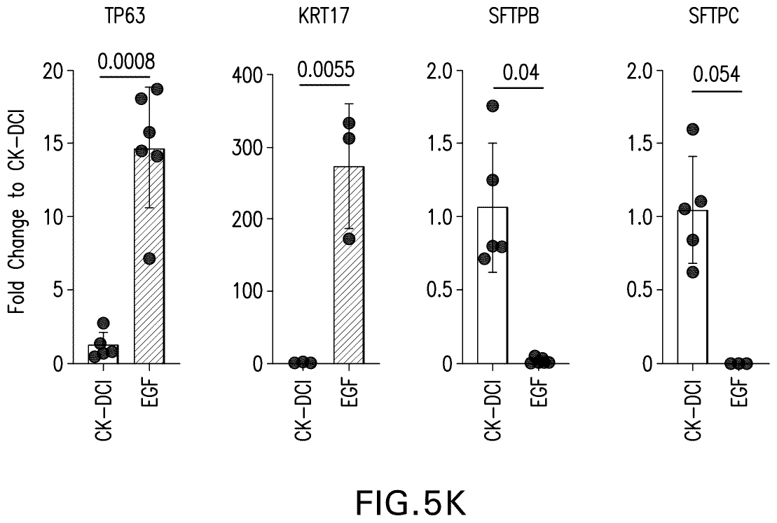

We next used empirical approaches to differentiate the cells into more mature proximal of distal lineages. We found that removing CHIR and KGF and blocking TGF-b signaling using a small molecule, SB, resulted in further upregulation of not only AT2, but also of AT1 markers (FIGS. 5a-5e). On the other hand, culture in 2D in the presence of feeders, ROCK inhibitor and EGF resulted in cells with phenotype consistent with airway BCs (FIGS. 5f-5k). Together, these data show that TLOs are multipotential.

### 4. TLOs in Disease Modeling

### 4.1. Idiopathic Pulmonary Fibrosis

Idiopathic pulmonary fibrosis (IPF) is a lethal disease for which there is currently no curative therapy, except for lung transplantation. While age and certain exposures are risk factors, up to 20% of patients show familial predisposition.^{35,36} Rare and common variants, some of which need functional validation, have been linked to IPF susceptibility in linkage, candidate gene, GWAS, Mendelian randomization, and next generation sequencing studies.³⁷⁻⁴¹ These studies revealed two classes of molecularly distinct etiologies that underly susceptibility to IPF: mutation or variants in genes that maintain mitotic chromosome and telomere integrity,^{39,42} and in genes affecting surfactant production.⁴³ Since DNA damage responses and proteostasis are connected,⁴ the pathogenesis of IPF may converge on failure to maintain cellular quality control in surfactant-producing type 2 alveolar epithelial (AT2) cells, who devote their endosomal and proteostatic machinery to homeostasis of highly hydrophobic surfactant proteins.⁴³

A challenge to developing novel treatments is the availability of well-validated models. Many mouse models have no phenotypes unless challenged with bleomycin.⁴⁵ While these models are useful as they recapitulate increased susceptibility to AT2 injury and a fibrotic response, they are less amenable to higher throughput mechanistic studies, validation of genetic variants and discovery of therapeutic targets.

The need for more clinically relevant experimental models is highlighted by the recent discovery through scRNAseq of several types of the aforementioned novel putative progenitor cells, collectively called ‘transitional cells’, that are absent from murine lungs.⁴⁶⁻⁴⁸ Remodeled regions of IPF lungs also show aberrant expression of proximal markers, described as bronchiolization⁴⁹. Furthermore, a novel aberrant basaloid population specific to IPF has been identified, characterized by expression of KRT17, COL1A1 and other ECM components, and most airway basal cell (BC) markers, but lacking others such as KRT5 (KRT5⁻, KRT17⁺).^{47,50} All these populations are present in the TLOs described here. While the accumulation of these populations is typically viewed as reactive to AT2 injury and as indicative of abnormal repair, we hypothesize that they may play a causative role in IPF. Under this hypothesis, functional impairment of these cells may in fact be one cause of the aberrant fibrotic response to injury observed in IPF. If true, then mutations observed in familial IPF would be expected to impair the function and phenotypes of these transitional cell types. On the other hand, if mutations involved in familial IPF do not affect these populations in isolation, then they must be truly reactive in nature.

### 4.2. Disease Models

We used the TLO model to determine if transitional cell populations are affected by mutations implicated in IPF. We focused on two models. The first is Hermansky-Pudlak Syndrome (HPS). We developed a lung organoid model derived from human pluripotent stem cells, where introduction of mutations in genes associated with HPS resulted in fibrotic changes only in organoids with mutations associated with fibrosis in patients.^{28,52,53} These studies led to the identification of IL-11 as a validated therapeutic target.⁵² We could also validate the effect of an approved treatment that slows disease progression but is not curative, perfinidone.⁵⁴ The second model is deletion of ABCA3. ABCA3 encodes a lipid transporter that is essential for production of mature surfactant.⁵⁵ ABCA3 mutations are associated with a wide spectrum of clinical manifestations, but most commonly causes childhood ILD. However, ABCA3 mutations can be associated with progressive fibrosis.^{56,57}

### 4.3. Gene Expression Phenotypes

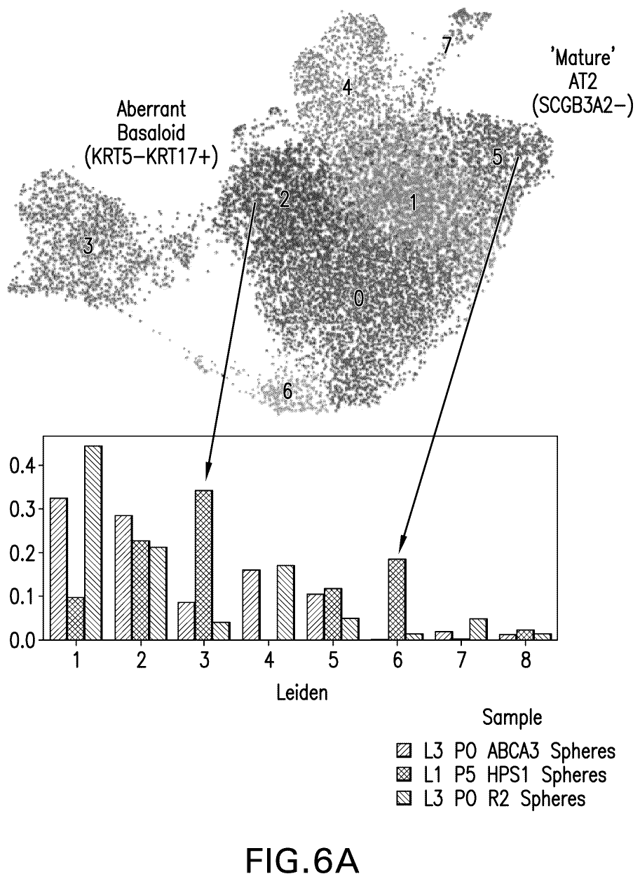

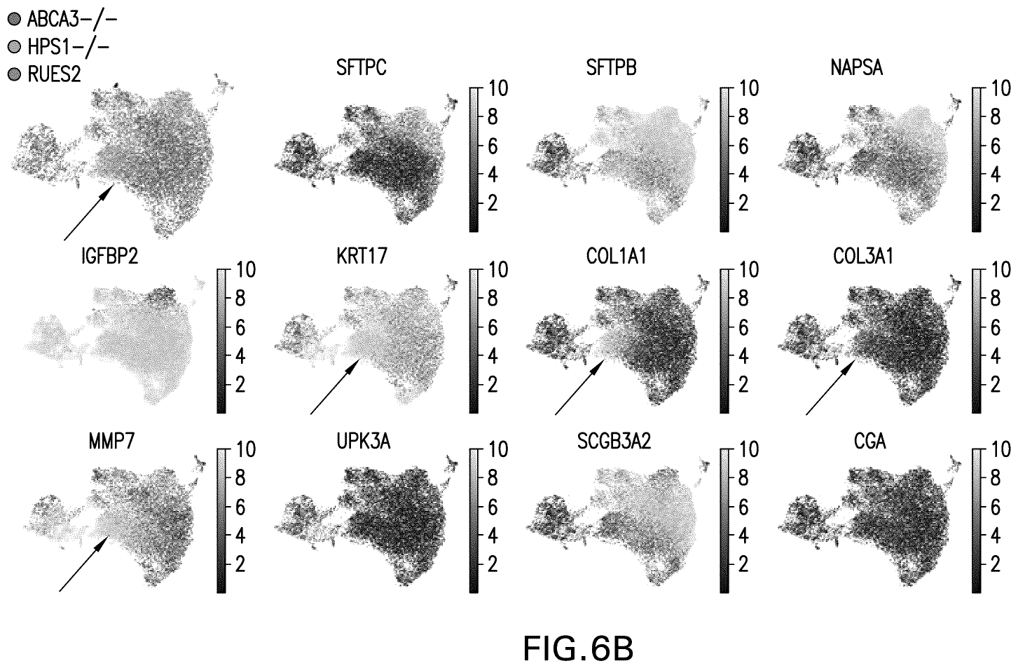

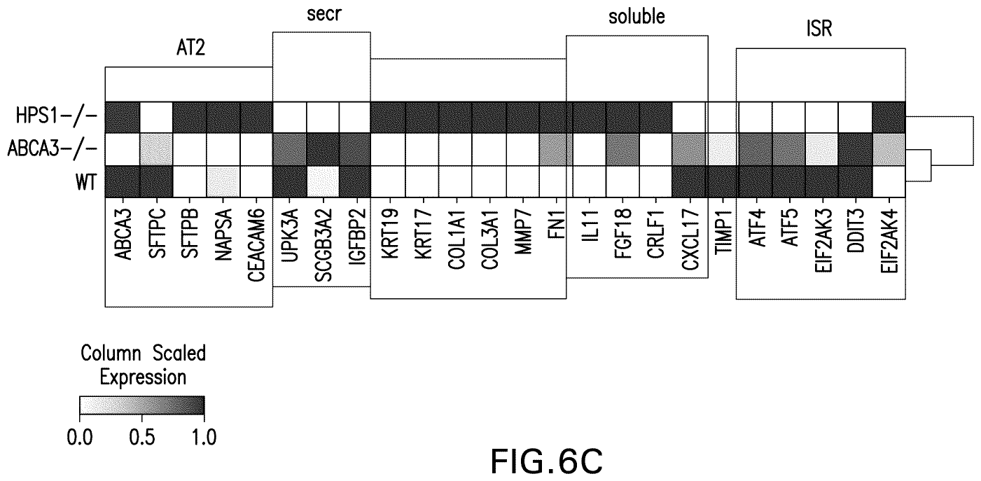

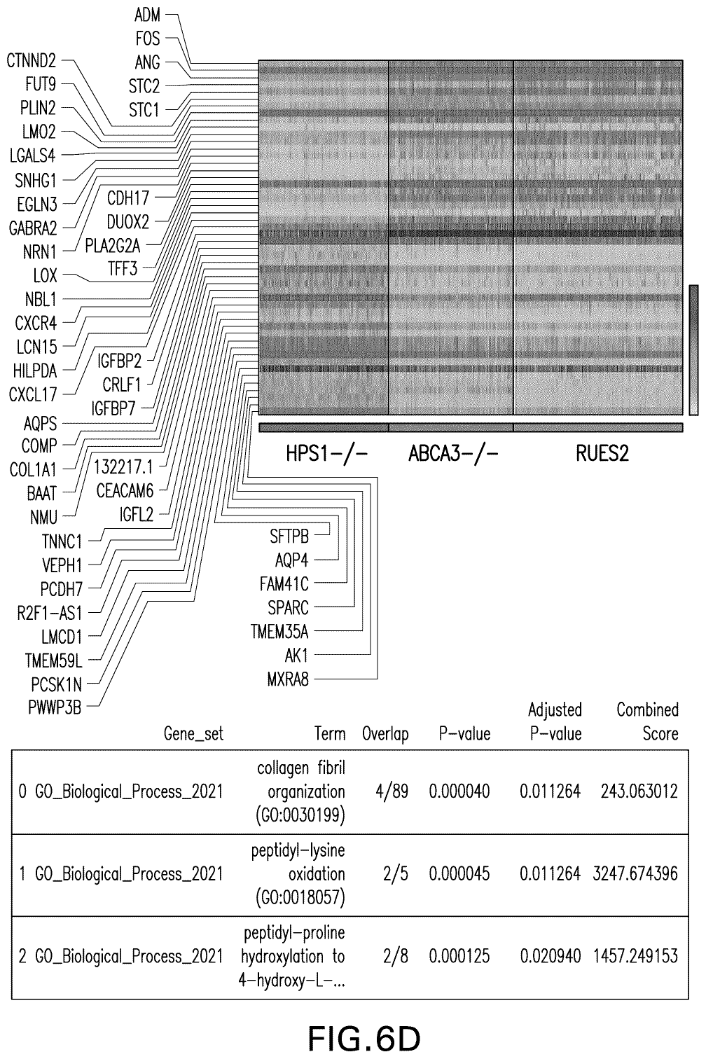

To examine if TLOs were affected by mutations involved in IPF, and could be used to gain insight into pathogenesis, we generated TLOs from HPS1^{-/- 58} and ABCA3^{-/-} mutant ESCs, produced using CRIPSR/Cas9 targeting, and performed scRNAseq. HPS1^{-/-} TLOs showed enrichment in two populations, that we identified as aberrant basaloid cells (a cell type prevalent in IPF lungs),^{47,50} and in mature AT2 cells (since they did not express SCGB3A2, a cell type not observed in wildtype (wt) TLOs) (FIGS. 6a, 6b). These AT2 cells (FIG. 6c), however, did show reduced expression of SFTPC, indicating abnormal differentiation. More focused analysis showed again increased expression of AT2 markers but reduced expression of SFTPC, increased expression of aberrant basaloid markers, but also dysregulated expression of genes involved in the integrated stress response (ISR) and ER stress (FIG. 6c). In addition, several soluble molecules were differentially regulated, including IL11, CRLF1, FGF18 and CXCL17 (FIG. 6c). Pseudo-bulk analysis revealed pathways related to collagen deposition as the most differentially expressed (FIG. 6d). ABCA3^{-/-} TLOs were very similar to WT, but expressed less ABCA3, as expected, and also showed some dysregulation of ISR/ER stress genes (FIG. 6a-d).

### 4.4. Defects in Differentiation Capacity

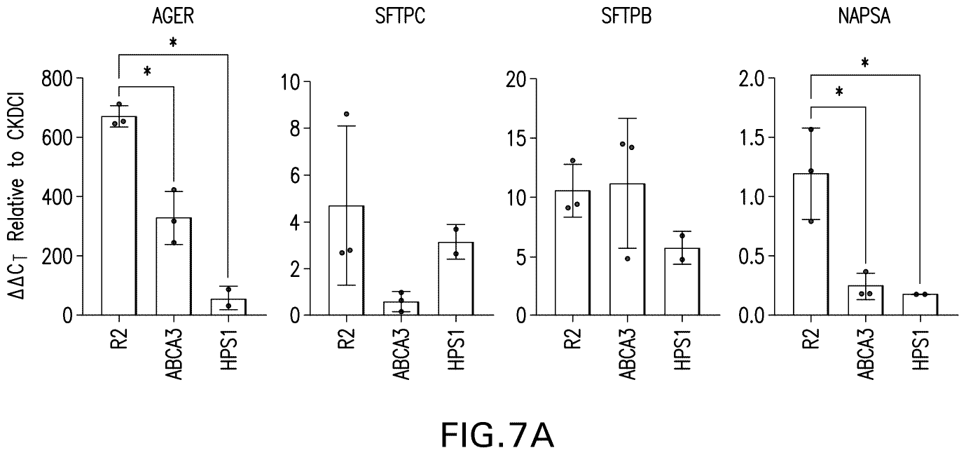

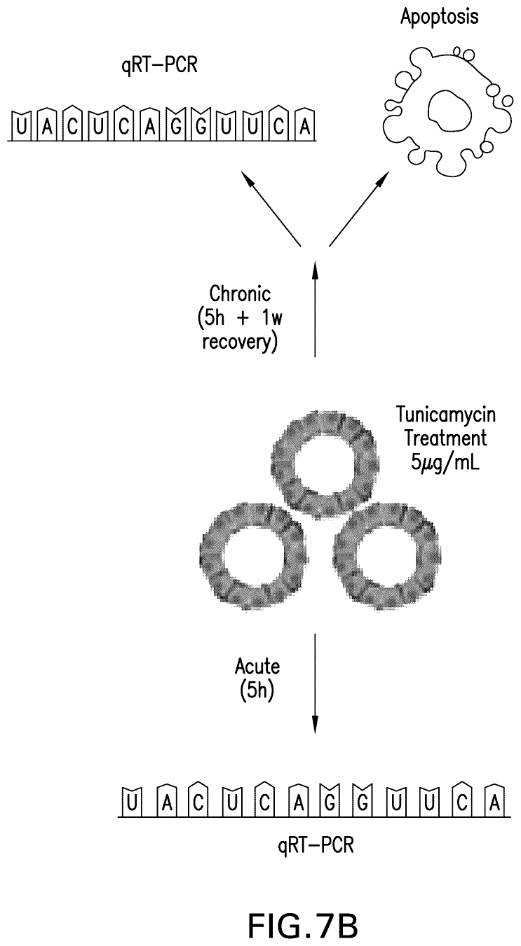

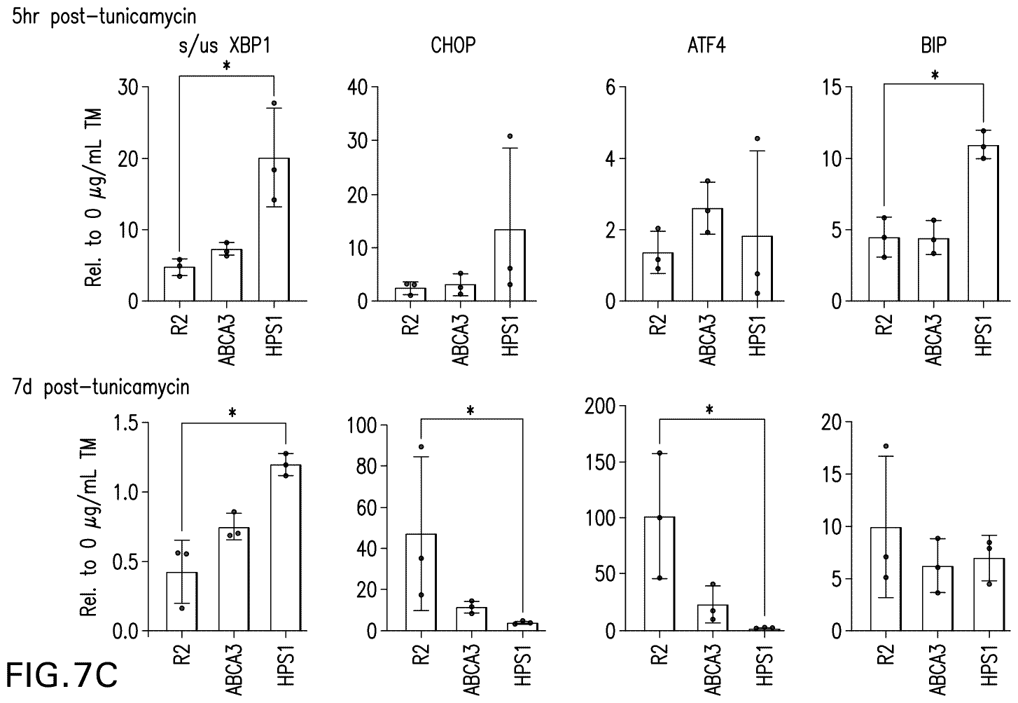

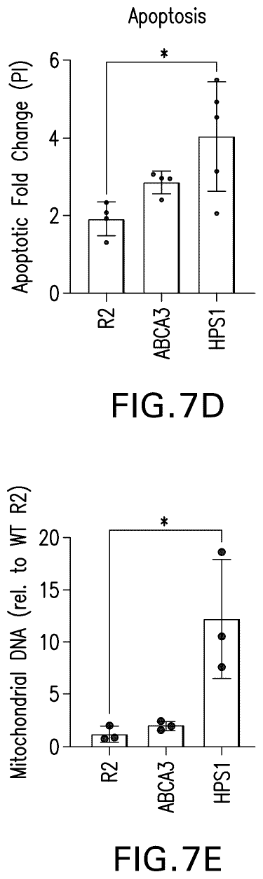

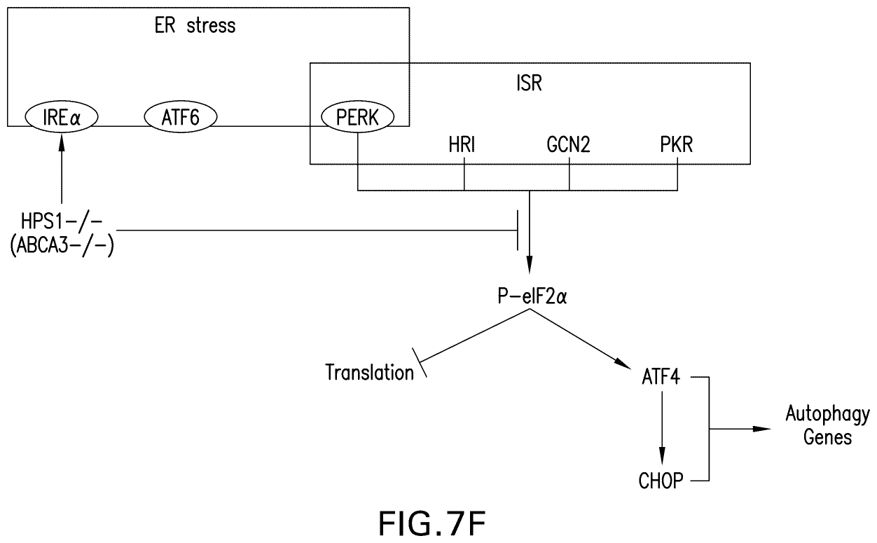

HPS1^{-/-} TLOs were significantly less capable of differentiation towards AT1 cells in the presence of DCI+SB (FIG. 5a-e) as measured by AGER expression, while ABCA3^{-/-} were as capable as WT (FIG. 7a). Differentiation towards AT2 cells did not appear impaired, with similar SFTPC, and SFTPB induction in all three genotypes, although expression of NAPSA, another AT2 marker, was significantly decreased in HPS1^{-/-} and ABCA3^{-/-} TLOs (FIG. 7a). Overall, these studies suggest impaired differentiation in mutant TLOs.

### 4.5. Cellular Stress Responses

Given the importance of ER stress in fibrotic disease,⁵⁹ we assessed the unfolded protein response of the ER (UPR^{ER}) activation in mutant TLOs. During ER stress, BIP binds to misfolded proteins in the ER, and then activates the three sensors of the ER stress pathway, IRE1α, PERK, and ATF6.⁶⁰ Activation of IRE1α causes splicing of X-box protein 1 (XBP1), which then promotes UPR gene expression.⁶¹ PERK phosphorylation by BIP inhibits protein translation and activates ATF4 expression to promote cell maintenance via pathways such as adaptive antioxidant responses and autophagy.⁶² And lastly, BIP dissociation from ATF6 allows ATF6 to traffic to the Golgi and then into the nucleus where it promotes transcription of ER chaperones⁶³. All three pathways can activate CHOP, a downstream effector protein that, amongst other functions, can trigger apoptosis.⁶⁴

At baseline, no ER stress was observed in all three lines (not shown). We challenged for 5 hours with tunicamycin, a protein glycosylation inhibitor that prevents protein maturation and causes ER stress.⁶⁵ and measured ER stress components after 5 hrs, upon removal tunicamycin, and after 1 week (FIG. 7b). HSP1^{-/-} TLOs showed significantly increased expression of BIP and of XBP1 splicing, indicating increased activation of the IRE1α-XBP1 axis (FIG. 7c). One week after removal of tunicamycin (trace tunicamycin likely remained in the Matrigel), WT (R2) TLO XBP1 and BIP expression returned to below baseline, and expression of BIP, CHOP and ATF4 were strikingly increased. However, HPS1^{-/-} TLOs maintained elevated splicing of XBP1 but entirely failed to upregulate CHOP and ATF4 (FIG. 7c). A similar response, though less pronounced, was observed in ABCA3^{-/-} TLOs (FIG. 7c). Propidium iodide staining showed increased apoptosis in HPS1^{-/-} TLOs at that time point (FIG. 7d). These data suggest that HPS1^{-/-} TLOs are more sensitive to ER stress, particularly over-activating the IRE1α-XBP1 axis, but appear unable to activate at least one other arm of the UPR^{ER}. Of particular interest is ATF4, as ATF4 is an effector protein implicated not only in the UPR^{ER} via PERK phosphorylation but also in the integrated stress response (ISR)⁶⁶.

As autophagy and mitophagy have been implicated in IPF as well,⁴³ and are induced by the ISR.⁶⁷ we determined mitochondrial mass as an indirect measure of mitophagy. qRT-PCR comparing the relative quantitative amount of a mitochondrial gene (tRNA-Leu) to genomic gene (82-microglobulin) across genotypes showed elevated mitochondrial mass in HPS1^{-/-} TLOs (FIG. 7e), suggesting a defect in mitophagy, and therefore likely in autophagy.

### 4.6. Disease Modeling: Conclusion

These data show that transitional cell populations are not merely reactive to AT2 injury, but rather, that they themselves display functional defects that may induce fibrosis. These abnormalities involve gene expression, response to proteotoxic stress, and possibly mitophagy/autophagy. Except for a more mildly abnormal response to proteotoxic stress, these phenotypes were undetectable in ABCA3^{-/-} TLOs, possibly corresponding to the fact that ABCA3 mutations are less strongly associated with fibrosis, although IPF patients with ABCA3 mutations have been described.⁵⁷ Taken together, it is not excluded, and even likely, that transitional cells play a key role in the pathogenesis of IPF, and may be at the basis of the aberrant, fibrotic injury response observed in this disease.

We show that TLOs revealed a defective ISR as a potential key driver of IPF. The ISR is the most important final common pathway of cellular stress.⁶⁸ In the ISR, a variety of signaling pathways, including ER stress, mitochondrial stress, viral infection and cytoplasmic proteotoxicity, phosphorylate eIF2a (P-eIF2a) which causes binding of the eIF2 guanine exchange factor, eIF2B, as a non-competitive inhibitor rather than as an activator of eIF2. This leads to disruption of the ternary complex (Met-tRNA, GTP and eIF2) required for translation initiation. Translation inhibition is selective however, as translation of at least some proteins with inhibitory upstream ORFs (uORFs), capable of AUG-independent translation initiation, are not or less affected by the ISR, prime among those ATF4, which induces a broad array of genes involved in the stress response, including CHOP (FIG. 7f).⁶⁸ Our findings therefore suggest that a defective ISR in HSP1^{-/-}, and to a lesser extent in ABCA3^{-/-} TLOs, may be a driver fibrosis that renders alveolar progenitors exquisitely sensitive to stress. These findings offer important avenues for drug discovery and validation.

### 5. Conclusion

We conclude that TLOs are an ideal model for studying novel cell types involved in human lung regeneration and for mechanistic studies and drug testing in IPF models, offering the advantage of a malleable, reductionist in vitro model and the potential to test the causal role of multiple genetic variants as well as individualized drug testing for precision medicine approaches in the treatment of IPF.

### Methods

### Human Lung Samples

De-identified normal human lung samples were provided by the Herbert Irving Comprehensive Cancer Center Molecular Pathology Shared Resource Tissue Bank core under IRB-AAAT8682.
Maintenance of hPSCs
Before differentiation, Rockefeller University Embryonic Stem Cell Line 2 (RUES2, passage 24-32) or mRNAi PSC lines (generated using mRNA transfection and obtained from the Mount Sinai Stem Cell Core, NY) were maintained on mouse embryonic fibroblasts (MEFs) plated at 22,500 cells/cm². Cells were cultured in hESC maintenance media (DMEM/F12 (ThermoFisher, Carlsbad, CA), 20% Knock-out serum, (Stem Cell Technologies, Vancouver, BC), 0.1 mM β-mercaptoethanol (Sigma-Aldrich, Burlington, MA), 0.2% Primocin (InvivoGen, San Diego, CA), 20 ng/ml FGF2 (R&D Systems/Biotechne, Minneapolis, MN), and 1% Glutamax (ThermoFisher)) which was changed daily. hPSCs were passaged every 3-4 days with Accutase (Innovative Cell Technologies, San Diego, CA) at least two times before differentiation, washed and replated at a dilution of 1:24. Cultures were maintained in a humidified 5% CO₂ atmosphere at 37° C. Lines are karyotyped and verified for Mycoplasma contamination using PCR (InVivoGen) every 6 months.
Generation of hESC-Derived Lung Organoids
The hESC-derived human lung organoids were generated as described previously.²⁸ Briefly, MEFs were depleted by passaging 5-7×10⁶ hESCs onto Matrigel (Corning, Corning, NY) coated 10-cm dish. Cells were maintained in hESC media in a humidified 5% CO₂ atmosphere at 37° C. After 24 hours, cells were detached with 0.05% Trypsin/EDTA (ThermoFisher) and distributed to the 6-well low attachment plate containing primitive streak/embryoid body media (10 μM Y-27632 (Tocris/Biotehne, Minneapolis, MN), 3 ng/ml BMP4 (R&D/Biotechne)) to allow embryoid body formation. Embryoid bodies were fed every day with fresh endoderm induction media (10 μM Y-27632, 0.5 ng/ml BMP4, 2.5 ng/ml FGF2 and 100 ng/ml ActivinA (R&D/Biotechne)) and maintained in a humidified 5% CO₂/5% O₂ atmosphere at 37° C. Endoderm yield efficiency was determined by dissociating embryoid bodies and evaluating CXCR4 and c-KIT (Biolegend, San Diego, CA) co-expression by flow cytometry on day 4. Cells used in all experiments had >90% endoderm yield and were plated on 0.2% fibronectin-coated wells (R&D/Biotechne) at a density of 80,000 cells/cm². Cells were incubated in Anteriorization media-1 (100 ng/ml Noggin (R&D/Biotechne) and 10 μM SB431542 (Tocris)) for 24 hours, followed by Anteriorization media-2 (10 μM SB431542 (Tocris) and 1 μM IWP2 (Tocris)) for another 24 hours. At the end of anterior foregut endoderm induction, cells were switched to Ventralization/Branching media (3 μM CHIR99021 (Tocris), 10 ng/ml FGF10 (R&D/Biotechne), 10 ng/ml rhKGF (recombinant human KGF, R&D/Biotechne), 10 ng/ml BMP4 and 50 nM all-trans Retinoic acid (Tocris)) for 48 hours and three-dimensional clump formation was observed. The adherent clumps were detached by gentle pipetting and transferred to the low-attachment plate, where they folded into lung bud organoids as early as d10-d12 (LBOs). Branching media was changed every other day until d20-d25 and LBOs were embedded in 100% Matrigel in 24-well transwell (BDFalcon, Franklin Lakes, NJ) inserts. Branching media was added after Matrigel solidified and changed every 2-3 days to facilitate proper growth into lung organoids. Culture of embedded organoids can be kept for more than 6 months.

### Generation of Transitional Lung Organoids (TLOs)

Matrigel embedded lung organoids can be used for TLO generation when they reach d42 of development. Media was removed from the transwell and 1 ml of 2 mg/ml dispase (Corning) was added to release lung organoid from the Matrigel for 30-45 minutes in normoxic incubator. The organoid was transferred to a 15 ml conical tube and washed with stop media (DMEM (Corning), 5% FBS (Atlanta Biologicals; Flowery Branch, Georgia), 1% Glutamax (ThermoFisher)) to neutralize Dispase, then centrifuged at 200 μg for 5 minutes. The pellet was incubated with 1 ml of 0.05% Trypsin/EDTA in normoxic incubator for 10-12 minutes with occasional pipetting with a P1000 pipet tip. Single cell dissociation was verified using a bright field microscope. If a single cell suspension was not obtained after 12 minutes, cells were washed with stop media and incubated for additional 5 minutes with 0.05% Trypsin/EDTA. Cells were counted using a hemocytometer and 400 cells/μl of undiluted Matrigel were plated in a well of a 12-well non-tissue culture plate. The plate was placed in normoxic incubator for 30 minutes until Matrigel polymerized and 1 ml of CKDCI (3 μM CHIR 99021, 10 ng/ml rhKGF, 50 ng/ml dexamethasone (ThermoFisher), 0.1 mM 8-Bromoadenosine 3′,5′-cyclic monophosphate sodium salt (Tocris) and 0.1 mM 3-Isobutyl-1-methylxanthine (Sigma-Aldrich)) media was gently added using 10 ml serological pipette. Media was changed every 3 days. After 2-3 weeks, a TLO culture is established at that can be maintained by regular passaging for more than six months.

### RT-qPCR

Total RNA was extracted according to manufacturer's instructions using the Direct-zol™ RNA Microprep (Zymo Research, Irvine, CA), and 500 ng of total RNA was reverse transcribed using the qScript™ XLT cDNA SuperMix (Quantabio, Beverly, MA). Technical triplicates of 15 ml reaction (for use in Applied Biosystems QuantStudio7 384-well System, Waltham, MA) were prepared with 3 μl of diluted cDNA and run for 40 cycles. Relative gene expression was calculated based on the average cycle (Ct) value, normalized to GAPDH as the internal control and reported as fold change (2(−ddCT)).

### Immunofluorescence

After removal of media, a flat edge was used to carefully detach the Matrigel droplet with embedded TLOs from the bottom of the 12 well plate. The droplet was then transferred to an OCT histology mold and embedded in Tissue-Tek OCT Compound (Sakura Finetek, Torrance, CA). Frozen samples were cut on cryotome at the thickness of 10-12 mm and collected on adhesion microscope slides, air-dried and fixed in 4% paraformaldehyde, then washed 2 times for 5 minutes in 50 mM glycine to inactivate PFA, followed by washing in PBS. Samples were permeabilized for 10 minutes in 0.2% PBST (PBS+0.2% Triton X-100) and blocked by incubating in PBS containing 5% donkey serum, then incubated overnight in primary antibody in 0.2% Triton X-100 and 2% donkey serum. Next day, samples were washed three times in PBS and 1% donkey serum and incubated with secondary antibody (1:200) for 1 hour at room temperature. Nuclei were stained with DAPI (ThermoFisher) and sections were mounted with Mounting Reagent (DAKO, Santa Clara, CA) and cover-slipped. Samples were imaged using a Leica TCS SP8 Stellaris Laser scanning confocal microscope, and Leica DMi1 Inverted Phase Contrast Microscope (Leica Microsystems, Deerfield, IL).

### Flow Cytometry

Spheres embedded in Matrigel were released by incubating with Dispase for 30-60 minutes, then washed and dissociated into single cells with 0.05% Trypsin/EDTA in normoxic incubator for 10-12 minutes with occasional pipetting with P1000. The single cell suspension was stained in polystyrene round-bottom 12×75 mm tubes (BD Falcon). Primary HTII-280 antibody (Terrace Biotech, San Francisco, CA, 1:150 dilution) was added to 150 μl of cell suspension and incubated for 1 hour at the room temperature. Cells were washed two times with FACS buffer (PBS, 10% FBS and 1% sodium azide) and centrifuged for 5 minutes at 1400 rpm. Fluorochrome-labeled secondary antibody (Alexa Fluor 488 goat anti-mouse IgM) in diluted in FACS buffer at 1:100 ratio was added for 30 minutes in the dark. Cells are washed two times by centrifugation. Conjugated human EPCAM antibody (Biolegend) was added for 30 minutes. Cells were washed and resuspended in FACS buffer for flow cytometric analysis.

### Transmission Electron Microscopy

Transmission Electron Microscopy (TEM) was performed. TLOs were fixed with 2% paraformaldehyde and 2.5% glutaraldehyde in 0.1 μM sodium cacodylate buffer (pH7.2) for 2 hours and post-fixed with 1% osmium tetroxide for 1.5 hours at room temperature, then processed in a standard manner and embedded in EMbed 812 (Electron Microscopy Sciences, Hatfield, PA). Semi-thin sections were cut at 1 mm and stained with 1% Toluidine Blue to evaluate the quality of preservation and find the area of interest. Ultrathin sections (60 nm) were cut, mounted on copper grids and stained with uranyl acetate and lead citrate by standard methods. Stained grids were examined under Philips CM-12 electron microscope and photographed with a Gatan (4k×2.7k) digital camera (Gatan, Inc., Pleasanton, CA).

### Western Blot

Cultured cells embedded in Matrigel were released by incubating with Dispase (Corning) for 30 minutes. Cells were then washed, collected in PBS, and treated with lysis buffer (RIPA buffer, lx Roche Complete Protease inhibitor cocktail, and Roche PhosSTOP). Buffer-treated cells were mechanically lysed using a 25-gauge needle and left on ice for 30 minutes. Human lung samples were homogenized with stainless steel beads and left to lyse on ice in lysis buffer for 4-6 hours. Samples were then centrifuged at 15,000 μg for 10 minutes and the supernatant was collected and stored at −80° C. until analysis. Protein concentration was measured using Pierce BCA Protein Assay Kit (ThermoFisher). A total of 18 μg of TLO, hPSC, and human lung lysate were loaded for mature SPC analysis and a total of 5 μg of each were loaded for mature SPB analysis. Lysates were resolved on pre-cast NuPage 4-12% Bis-Tris gels (Invitrogen) and transferred to nitrocellulose membranes (Bio-Rad, Hercules, CA). The following primary antibodies were used to probe the blots: mature SPC (1:1000, Seven Hills Bioreagents, Cincinatti, OH), mature SPB (1:1000, Seven Hills Bioreagents), GAPDH (1:5000, Cell Signaling Technology). Species-specific secondary antibodies with HRP conjugates (Cell Signaling Technology, Danvers, MA) were used at 1:15,000 dilution. Blots were then treated with Pierce ECL Western Blotting Substrate and visualized using the Amersham ImageQuant 800 (Cytiva, Marlborough, MA).

### Cryopreservation of TLOs

Matrigel embedded spheres can be frozen and thawed for future use. 2-3 weeks after passaging of spheres, CK-DCI media was removed and 1 mL of Dispase (2 mg/mL) was used to break apart the Matrigel droplet. After 30-45 minutes in normoxic incubator, the spheres were transferred to a 15 mL conical and washed with wash media to neutralize the protease, then centrifuged at 200 g for 5 minutes. Wash media was aspirated, and the pellet was incubated with 1 mL of 0.05% Trypsin/EDTA in normoxic incubator for 10-12 minutes with occasional pipetting. Once cells are in single cell suspension, they were washed again with stop media, centrifuged at 200 g for 5 minutes, and the supernatant aspirated, followed by resuspension of the pellet in a small volume of CK-DCI and counted using a hemocytometer. To freeze down, a density of 500,000 to 1 million singe cells is ideal. Cells are resuspended in equal volumes of CK-DCI media and 2× DMSO freezing medium (Quality Biological, Gaitersburg, MD) and transferred to a cryovial. Vials are immediately place in a container that allows freezing rate of −1° C./min and placed in a −80° C. freezer. Next day, they are transferred to liquid nitrogen. To thaw, cryovial is places in a 37° C. water bath until thawed. Cells are transferred to a 15 mL conical tube and washed with stop media, centrifuged at 200 g for 5 minutes, and aspirated. The ideal initial reseeding density post-thaw is 800-1,600 cells per μL of undiluted Matrigel. Subsequent passages can be done at 400 cells per μL.
Single-Cell cDNA Library Preparation and scRNA-Seq
Viability of single cells was assessed using Trypan Blue staining, and debris-free suspensions of >80% viability were deemed suitable for single cell RNA Seq. Samples with lower viability were run with caution. Single-cell RNA-seq was performed on these samples using the Chromium platform (10× Genomics, Pleasanton, CA) with the 3′ gene expression (3′ GEX) V3 kit, using an input of ˜10,000 cells. Briefly, Gel-Bead in Emulsions (GEMs) were generated on the sample chip in the Chromium controller. Barcoded cDNA was extracted from the GEMs by Post-GEM RT-cleanup and amplified for 12 cycles. Amplified cDNA was fragmented and subjected to end-repair, poly A-tailing, adapter ligation, and 10×-specific sample indexing following the manufacturer's protocol. Libraries were quantified using Bioanalyzer (Agilent) and QuBit (Thermofisher) analysis and were sequenced in paired end mode on a NovaSeq instrument (Illumina, San Diego, CA) targeting a depth of 50,000-100,000 reads per cell.
scRNA-Seq Computational Analysis
Sequencing data were aligned and quantified using the Cell Ranger Single-Cell Software Suite (version 6.1.2, 10x Genomics) against the provided GRCh38 (Ensembl 98) human reference genome. All further computational analysis of scRNA-seq data was performed using R version 4.1.3 (https://www.R-project.org/) unless otherwise stated.

### QC and Processing

The aligned data was imported and processed using the R package Seurat v4.1.1⁶⁹. Quality control for doublets and low-quality cells was achieved through exclusion of cells with less than 500 or more than 9000 transcripts and those with a higher than 20% mitochondrial gene contribution, respectively. Additionally, transcripts were retained if they counted over 0 in more than 0.5% of all cells, otherwise excluded. Count data was then log-normalized and transcripts were scaled and centered, using built-in Seurat functions. Variable transcripts were calculated based on standardized feature values using observed mean and expected variance of a local polynomial regression model. On the resulting variable transcripts 50 principal components were computed, which in turn were used as input for uniform manifold approximation and projection (UMAP) dimensionality reduction. For clustering analysis, a shared nearest neighbor (SNN) graph was constructed and the modularity function optimized using the Leiden algorithm.

### Cell Type Annotation

Potential cell types present in our dataset were predicted through machine learning. In brief, a random forest classifier (SingleCellNet R package)⁷⁰ was trained on fully annotated published data by Murthy et al. and Haberman et al. and assessed on a withheld subset. This classifier was then applied to the current dataset and a matching cell type was predicted for each cell.

### RNA Velocity

RNA velocity was calculated in python using the packages Velocyto and scVelo, according to the developer's manual^{71,72} Briefly, ‘loom’ files containing both exon and intron information were created from aligned raw data using Velocyto. scVelo was then used to normalize the data and compute moments. Subsequently, RNA velocity was estimated and projected onto Seurat-derived UMAP coordinates. The length and coherence of the velocity vectors, which indicate differentiation speed and directional confidence, respectively, were calculated. Finally, a dynamical model was applied to analyze transcriptional states and cell-internal latent time and subsequently recompute RNA velocities. The latent time, based solely on the transcriptional dynamics of a cell, was thereby determined.

### REFERENCES

1. Habermann, A. C. et al. Single-cell RNA sequencing reveals profibrotic roles of distinct epithelial and mesenchymal lineages in pulmonary fibrosis. Sci. Adv. 6, eaba1972 (2020).
2. Basil, M. C. et al. Human distal airways contain a multipotent secretory cell that can regenerate alveoli. Nature 604, 120-126 (2022).
3. Kadur Lakshminarasimha Murthy, P. et al. Human distal lung maps and lineage hierarchies reveal a bipotent progenitor. Nature 604, 111-119 (2022).
4. Basil, M. C. et al. The Cellular and Physiological Basis for Lung Repair and Regeneration: Past, Present, and Future. Cell Stem Cell 26, 482-502 (2020).
5. Herriges, M. & Morrisey, E. E. Lung development: orchestrating the generation and regeneration of a complex organ. Development 141, 502-13 (2014).
6. Basil, M. C. et al. The Cellular and Physiological Basis for Lung Repair and Regeneration: Past, Present, and Future. Cell Stem Cell 26, 482-502 (2020).
7. Barkauskas, C. E. et al. Type 2 alveolar cells are stem cells in adult lung. J. Clin. Invest. 123, 3025-3036(2013).
8. Nabhan, A. N., Brownfield, D. G., Harbury, P. B., Krasnow, M. A. & Desai, T. J. Single-cell Wnt signaling niches maintain stemness of alveolar type 2 cells. Science 359, 1118-1123 (2018).
9. Zacharias, W. J. et al. Regeneration of the lung alveolus by an evolutionarily conserved epithelial progenitor. Nature 555, 251-255 (2018).
10. Jain, R. et al. Plasticity of Hopx(+) type I alveolar cells to regenerate type II cells in the lung. Nat. Commun. 6, 6727 (2015).
11. Kim, C. F. B. et al. Identification of bronchioalveolar stem cells in normal lung and lung cancer. Cell 121, 823-835 (2005).
12. Liu, Q. et al. Lung regeneration by multipotent stem cells residing at the bronchioalveolar-duct junction. Nat. Genet. 51, 728-738 (2019).
13. Kumar, P. A. et al. Distal airway stem cells yield alveoli in vitro and during lung regeneration following H1N1 influenza infection. Cell 147, 525-538 (2011).
14. Zuo, W. et al. p63(+)Krt5(+) distal airway stem cells are essential for lung regeneration. Nature 517, 616-620 (2015).
15. Choi, J. et al. Release of Notch activity coordinated by IL-1β signalling confers differentiation plasticity of airway progenitors via Fos12 during alveolar regeneration. Nat. Cell Biol. 23, 953-966 (2021).
16. Jeon, H. Y. et al. Airway secretory cell fate conversion via YAP-mTORC1-dependent essential amino acid metabolism. EMBO J. 41, e109365 (2022).
17. Vaughan, A. E. et al. Lineage-negative progenitors mobilize to regenerate lung epithelium after major injury. Nature 517, 621-625 (2015).
18. Kathiriya, J. J., Brumwell, A. N., Jackson, J. R., Tang, X. & Chapman, H. A. Distinct Airway Epithelial Stem Cells Hide among Club Cells but Mobilize to Promote Alveolar Regeneration. Cell Stem Cell 26, 346-358.e4 (2020).
19. Guha, A., Deshpande, A., Jain, A., Sebastiani, P. & Cardoso, W. V. Uroplakin 3a+ Cells Are a Distinctive Population of Epithelial Progenitors that Contribute to Airway Maintenance and Post-injury Repair. Cell Rep. 19, 246-254 (2017).
20. Rawlins, E. L. et al. The role of Scgb1a1+ Clara cells in the long-term maintenance and repair of lung airway, but not alveolar, epithelium. Cell Stem Cell 4, 525-534 (2009).
21. Melms, J. C. et al. A molecular single-cell lung atlas of lethal COVID-19. Nature 595, 114-119 (2021).
22. Adams, T. S. et al. Single-cell RNA-seq reveals ectopic and aberrant lung-resident cell populations in idiopathic pulmonary fibrosis. Sci. Adv. 6, eaba1983 (2020).
23. Street, K. et al. Slingshot: cell lineage and pseudotime inference for single-cell transcriptomics. BMC Genomics 19, 477 (2018).
24. Bergen, V., Lange, M., Peidli, S., Wolf, F. A. & Theis, F. J. Generalizing RNA velocity to transient cell states through dynamical modeling. Nat. Biotechnol. 38, 1408-1414 (2020).
25. Yamamoto, Y. et al. Long-term expansion of alveolar stem cells derived from human iPS cells in organoids. Nat. Methods 14, 1097-1106 (2017).
26. Jacob, A. et al. Derivation of self-renewing lung alveolar epithelial type II cells from human pluripotent stem cells. Nat Protoc 14, 3303-3332 (2019).
27. Jacob, A. et al. Differentiation of Human Pluripotent Stem Cells into Functional Lung Alveolar Epithelial Cells. Cell Stem Cell 21, 472-488.e10 (2017).
28. Chen, Y.-W. et al. A three-dimensional model of human lung development and disease from pluripotent stem cells. Nat. Cell Biol. 19, 542-549 (2017).
29. Olmeda, B., Martinez-Calle, M. & Pérez-Gil, J. Pulmonary surfactant metabolism in the alveolar airspace: Biogenesis, extracellular conversions, recycling. Ann. Anat. Anat. Anz. Off Organ Anat. Ges. 209, 78-92 (2017).
30. Brehier, A. & Rooney, S. A. Phosphatidylcholine synthesis and glycogen depletion in fetal mouse lung: developmental changes and the effects of dexamethasone. Exp. Lung Res. 2, 273-287 (1981).
31. Chi, E. Y. The ultrastructural study of glycogen and lamellar bodies in the development of fetal monkey lung. Exp. Lung Res. 8, 275-289 (1985).
32. Sun, X. et al. A census of the lung: CellCards from LungMAP. Dev. Cell 57, 112-145.e2 (2022).
33. Adams, T. S. et al. Single-cell RNA-seq reveals ectopic and aberrant lung-resident cell populations in idiopathic pulmonary fibrosis. Sci. Adv. 6, eaba1983 (2020).
34. Gotoh, S. et al. Generation of alveolar epithelial spheroids via isolated progenitor cells from human pluripotent stem cells. Stem Cell Rep. 3, 394-403 (2014).
35. Wijsenbeek, M. & Cottin, V. Spectrum of Fibrotic Lung Diseases. N. Engl. J. Med. 383, 958-968 (2020).
36. Renzoni, E. A., Poletti, V. & Mackintosh, J. A. Disease pathology in fibrotic interstitial lung disease: is it all about usual interstitial pneumonia?The Lancet 398, 1437-1449 (2021).
37. Mathai, S. K., Newton, C. A., Schwartz, D. A. & Garcia, C. K. Pulmonary fibrosis in the era of stratified medicine. Thorax 71, 1154-1160 (2016).
38. Wang, Y. et al. Genetic defects in surfactant protein A2 are associated with pulmonary fibrosis and lung cancer. Am. J. Hum. Genet. 84, 52-59 (2009).
39. Zhang, D. et al. Rare and Common Variants in KIF15 Contribute to Genetic Risk of Idiopathic Pulmonary Fibrosis. Am. J. Respir. Crit. Care Med. 206, 56-69 (2022).
40. Sutton, R. M. et al. Rare surfactant-related variants in familial and sporadic pulmonary fibrosis. Hum. Mutat. 43, 2091-2101 (2022).
41. Diaz de Leon, A. et al. Subclinical lung disease, macrocytosis, and premature graying in kindreds with telomerase (TERT) mutations. Chest 140, 753-763 (2011).
42. Alder, J. K. & Armanios, M. Telomere-mediated lung disease. Physiol. Rev. 102, 1703-1720 (2022).
43. Katzen, J. & Beers, M. F. Contributions of alveolar epithelial cell quality control to pulmonary fibrosis. J Clin Invest (2020) doi:10.1172/jci139519.
44. Gonzilez-Quiroz, M. et al. When Endoplasmic Reticulum Proteostasis Meets the DNA Damage Response. Trends Cell Biol. 30, 881-891 (2020).
45. Tashiro, J. et al. Exploring Animal Models That Resemble Idiopathic Pulmonary Fibrosis. Front. Med. 4, 118 (2017).
46. Kadur Lakshminarasimha Murthy, P. et al. Human distal lung maps and lineage hierarchies reveal a bipotent progenitor. Nature 604, 111-119 (2022).
47. Habermann, A. C. et al. Single-cell RNA sequencing reveals profibrotic roles of distinct epithelial and mesenchymal lineages in pulmonary fibrosis. Sci. Adv. 6, eaba1972 (2020).
48. Basil, M. C. et al. Human distal airways contain a multipotent secretory cell that can regenerate alveoli. Nature 604, 120-126 (2022).
49. Chilosi, M. et al. Abnormal Re-epithelialization and Lung Remodeling in Idiopathic Pulmonary Fibrosis: The Role of ΔN-p63. Lab. Invest. 82, 1335-1345 (2002).
50. Adams, T. S. et al. Single-cell RNA-seq reveals ectopic and aberrant lung-resident cell populations in idiopathic pulmonary fibrosis. Sci. Adv. 6, eaba1983 (2020).
51. Seward, S. L., Jr & Gahl, W. A. Hermansky-Pudlak Syndrome: Health Care Throughout Life. Pediatrics 132, 153-160 (2013).
52. Strikoudis, A. et al. Modeling of Fibrotic Lung Disease Using 3D Organoids Derived from Human Pluripotent Stem Cells. Cell Rep. 27, 3709-3723.e5 (2019).
53. Matkovic Leko, I. et al. A distal lung organoid model to study interstitial lung disease, viral infection and human lung development. Nat. Protoc. (2023) doi:10.1038/s41596-023-00827-6.
54. Johannson, K. A., Chaudhuri, N., Adegunsoye, A. & Wolters, P. J. Treatment of fibrotic interstitial lung disease: current approaches and future directions. Lancet Lond. Engl. 398, 1450-1460 (2021).
55. Bullard, J. E., Wert, S. E., Whitsett, J. A., Dean, M. & Nogee, L. M. ABCA3 Mutations Associated with Pediatric Interstitial Lung Disease. Am. J. Respir. Crit. Care Med. 172, 1026 (2005).
56. Beers, M. F. & Mulugeta, S. The Biology of the ABCA3 Lipid Transporter in Lung Health and Disease. Cell Tissue Res. 367, 481-493 (2017).
57. Klay, D., Platenburg, M. G. J. P., van Rijswijk, R. H. N. A. J., Grutters, J. C. & van Moorsel, C. H. M. ABCA3 mutations in adult pulmonary fibrosis patients: a case series and review of literature. Curr. Opin. Pulm. Med. 26, 293-301 (2020).
58. Chen, Y.-W. et al. A three-dimensional model of human lung development and disease from pluripotent stem cells. Nat. Cell Biol. 19, 542-549 (2017).
59. Kropski, J. A. & Blackwell, T. S. Endoplasmic reticulum stress in the pathogenesis of fibrotic disease. J. Clin. Invest. 128, 64-73 (2018).
60. Bertolotti, A., Zhang, Y., Hendershot, L. M., Harding, H. P. & Ron, D. Dynamic interaction of BiP and ER stress transducers in the unfolded-protein response. Nat. Cell Biol. 2, 326-332 (2000).
61. Hollien, J. et al. Regulated Irel-dependent decay of messenger RNAs in mammalian cells. J. Cell Biol. 186, 323-331 (2009).
62. Carrara, M., Prischi, F., Nowak, P. R., Kopp, M. C. & Ali, M. M. Noncanonical binding of BiP ATPase domain to Irel and Perk is dissociated by unfolded protein CH1 to initiate ER stress signaling. eLife 4, e03522 (2015).
63. Ye, J. et al. E R Stress Induces Cleavage of Membrane-Bound ATF6 by the Same Proteases that Process SREBPs. Mol. Cell 6, 1355-1364 (2000).
64. Kim, I., Xu, W. & Reed, J. C. Cell death and endoplasmic reticulum stress: disease relevance and therapeutic opportunities. Nat. Rev. Drug Discov. 7, 1013-1030 (2008).
65. Azim, M. & Surani, H. Glycoprotein synthesis and inhibition of glycosylation by tunicamycin in preimplantation mouse embryos: Compaction and trophoblast adhesion. Cell 18, 217-227 (1979).
66. Costa-Mattioli, M. & Walter, P. The integrated stress response: From mechanism to disease. Science 368, eaat5314 (2020).
67. Katzen, J. & Beers, M. F. Contributions of alveolar epithelial cell quality control to pulmonary fibrosis. J. Clin. Invest. 130, 5088-5099 (2020).
68. Costa-Mattioli, M. & Walter, P. The integrated stress response: From mechanism to disease. Science 368, eaat5314 (2020).
69. Butler, A., Hoffman, P., Smibert, P., Papalexi, E. & Satija, R. Integrating single-cell transcriptomic data across different conditions, technologies, and species. Nat. Biotechnol. 36, 411-420 (2018).
70. Tan, Y. & Cahan, P. SingleCellNet: A Computational Tool to Classify Single Cell RNA-Seq Data Across Platforms and Across Species. Cell Syst. 9, 207-213.e2 (2019).
71. Bergen, V., Lange, M., Peidli, S., Wolf, F. A. & Theis, F. J. Generalizing RNA velocity to transient cell states through dynamical modeling. Nat. Biotechnol. 38, 1408-1414 (2020).
72. La Manno, G. et al. RNA velocity of single cells. Nature 560, 494-498 (2018).

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

What is claimed is:

1. A method for generating lung progenitor cells, the method comprising:
(a) producing anterior foregut endoderm cells from mammalian pluripotent stem cells (PSCs);
(b) culturing the anterior foregut endoderm cells in a suspension culture comprising a glycogen synthase kinase (GSK) inhibitor, a bone morphogenic protein (BMP) agonist, one or more FGF agonists, and retinoic acid, to generate at least one lung bud organoid (LBO);
(c) culturing the LBO in a three-dimensional (3D) matrix in presence of a GSK inhibitor, a BMP agonist, one or more FGF agonists, and retinoic acid, to form a branched LBO (BLBO); and
(d) dissociating the LBO or BLBO, and culturing the dissociated LBO or BLBO in a 3D matrix in presence of a GSK3 inhibitor, an FGF agonist, a corticosteroid, a 3′,5′-cyclic adenosine monophosphate (cAMP) pathway activator, and a phosphodiesterase (PDE) inhibitor.

2. The method of claim 1, wherein the GSK inhibitor is CHIR99021.

3. The method of any preceding claim, wherein in step (d) the GSK inhibitor is at a concentration ranging from about 1 μM to about 10 μM.

4. The method of any preceding claim, wherein in step (d) the GSK inhibitor is at a concentration of about 3 μM.

5. The method of any preceding claim, wherein the one or more FGF agonists are FGF10 and keratinocyte growth factor (KGF).

6. The method of any preceding claim, wherein the FGF agonist is KGF.

7. The method of any preceding claim, wherein the FGF agonist is at a concentration ranging from about 5 ng/ml to about 20 ng/ml.

8. The method of any preceding claim, wherein the FGF agonist is at a concentration of about 10 ng/ml.

9. The method of any preceding claim, wherein the corticosteroid is dexamethasone.

10. The method of any preceding claim, wherein the corticosteroid is at a concentration ranging from about 100 nM to about 150 nM.

11. The method of any preceding claim, wherein the corticosteroid is at a concentration of about 127 nM.

12. The method of any preceding claim, wherein the cAMP pathway activator is cAMP or 8-bromo-cAMP.

13. The method of any preceding claim, wherein the cAMP pathway activator is at a concentration ranging from about 0.05 mM to about 0.2 mM.

14. The method of any preceding claim, wherein the cAMP pathway activator is at a concentration of about 0.1 mM.

15. The method of any preceding claim, wherein the PDE inhibitor is 3-isobutyl-1-methylxanthine.

16. The method of any preceding claim, wherein the PDE inhibitor is at a concentration ranging from about 0.05 mM to about 0.2 mM.

17. The method of any preceding claim, wherein the PDE inhibitor is at a concentration of about 0.1 mM.

18. The method of any preceding claim, wherein the BMP agonist is BMP4.

19. The method of any preceding claim, wherein the BMP agonist is BMP4, the one or more FGF agonists are KGF and FGF10, and wherein KGF, FGF10, and/or BMP4 are at a concentration of about 10 ng/ml.

20. The method of any preceding claim, wherein retinoic acid is at a concentration of about 50 nM.

21. The method of any preceding claim, wherein in step (d) the LBO or BLBO is dissociated to single cells.

22. The method of any preceding claim, wherein the lung progenitor cells are in the form of lung organoids.

23. The method of any preceding claim, wherein the lung progenitor cells comprise type 0 alveolar epithelial (AT0) cells, terminal respiratory bronchiole stem cells (TRB-SCs) and distal basal cells (BCs).

24. The method of claim 23, wherein the lung progenitor cells further comprise neuroendocrine cells.

25. The method of any preceding claim, wherein the lung progenitor cells comprise SCGB3A2⁺SFTPB⁺SFTPC⁺ cells, SFTPB⁺SCGB3A2⁺SFTPC⁻ cells, and SFPTB⁺SCGB3A2⁻ cells.

26. The method of any preceding claim, wherein the LBO comprises (i) lung epithelial cells expressing FOXA2, FOXA1, NKX2.1 and EPCAM, and (ii) mesenchymal progenitors expressing PDGFRa, CD90, TBX4 and HOXA5.

27. The method of any preceding claim, wherein the 3D matrix is a solubilized basement membrane preparation from the Engelbreth-Holm-Swarm (EHS) mouse sarcoma.

28. The method of any preceding claim, wherein the mammalian pluripotent stem cells (PSCs) are human pluripotent stem cells (hPSCs).

29. The method of any preceding claim, wherein the mammalian pluripotent stem cells (PSCs) are embryonic stem cells (ESCs) and/or induced pluripotent stem cells (iPSCs).

30. Lung progenitor cells generated by the method of any preceding claim.

31. The lung progenitor cells of claim 30, comprising type 0 alveolar epithelial (AT0) cells, terminal respiratory bronchiole stem cells (TRB-SCs) and distal basal cells (BCs).

32. The lung progenitor cells of claim 31, further comprising neuroendocrine cells.

33. The lung progenitor cells of any of claims 30-32, comprising SCGB3A2⁺SFTPB⁺SFTPC⁺cells, SFTPB⁺SCGB3A2⁺SFTPC⁻ cells, and SFPTB⁺SCGB3A2⁻ cells.

34. A cell population comprising lung progenitor cells generated by the method of any of claims 1-29.

35. The cell population of claim 34, in the form of a lung organoid.

36. Lung progenitor cells generated in vitro, comprising type 0 alveolar epithelial (AT0) cells, terminal respiratory bronchiole stem cells (TRB-SCs) and distal basal cells (BCs).

37. The lung progenitor cells of claim 36, further comprising neuroendocrine cells.

38. Lung progenitor cells generated in vitro, comprising SCGB3A2⁺SFTPB⁺SFTPC⁺ cells, SFTPB⁺SCGB3A2⁺SFTPC⁻ cells, and SFPTB⁺SCGB3A2⁻ cells.

39. The lung progenitor cells of any of claims 36-38, expressing SFTPC and SFTPB.

40. The lung progenitor cells of any of claims 36-39, expressing EPCAM, NKX2.1 and SFTPB.

41. The lung progenitor cells of any of claims 36-40, expressing SCGB3A2.

42. The lung progenitor cells of any of claims 36-41, expressing mRNAs encoding SCGB3A2, SFTPC, SFTPB, ABCA3, LPCAT, NAPSA, SLC234A2, and LAMP3.

43. An artificial lung organoid generated in vitro, comprising type 0 alveolar epithelial (AT0) cells, terminal respiratory bronchiole stem cells (TRB-SCs) and distal basal cells (BCs).

44. The artificial lung organoid of claim 43, further comprising neuroendocrine cells.

45. An artificial lung organoid generated in vitro, comprising SCGB3A2⁺SFTPB⁺SFTPC⁺cells, SFTPB⁺SCGB3A2⁺SFTPC⁻ cells, and SFPTB⁺SCGB3A2⁻ cells.

46. A pharmaceutical composition comprising the lung progenitor cells of any of claims 30-33 and 36-42, the cell population of claim 34 or 35, or the artificial lung organoid of any of claims 43-45.

47. A method of treating a pulmonary disorder or injury in a subject in need thereof, the method comprising administering to the subject an effective amount of the lung progenitor cells of any of claims 30-33 and 36-42, the cell population of claim 34 or 35, or the artificial lung organoid of any of claims 43-45.

48. The method of claim 47, wherein the pulmonary disorder or injury is selected from the group consisting of: cystic fibrosis; emphysema; chronic obstructive pulmonary disease (COPD); pulmonary fibrosis; idiopathic pulmonary fibrosis (IPF); Hermansky-Pudlak Syndrome; hypersensitivity pneumonitis; sarcoidosis; asbestosis; autoimmune-mediated interstitial lung disease; pulmonary hypertension; lung cancer; acute lung injury (adult respiratory distress syndrome); respiratory distress syndrome of prematurity, chronic lung disease of prematurity (bronchopulmonary dysplasia); surfactant protein B deficiency, surfactant protein C deficiency, ABCA3 deficiency; NKX2.1 mutation; ciliopathies; congenital diaphragmatic hernia; pulmonary alveolar proteinosis; pulmonary hypoplasia; lung injury, and combinations thereof.

49. The method of claim 47 or 48, wherein the pulmonary disorder or injury is an interstitial lung disease or a congenital surfactant deficiency.

50. The method of any of claims 47-49, wherein the lung progenitor cells are non-syngeneic with the subject.

51. The method of any of claims 47-49, wherein the lung progenitor cells are syngeneic with the subject.

52. The method of any of claims 47-49, wherein the lung progenitor cells are allogeneic or xenogeneic with the subject.

53. A method of screening an agent for pharmacological or toxicological activity, the method comprising:
(a) administering an agent to the lung progenitor cells of any of claims 30-33 and 36-42, the cell population of claim 34 or 35, or the artificial lung organoid of any of claims 43-45; and
(b) assaying at least one pharmacological or toxicological response from at least one cell of the lung progenitor cells, the cell population, or the artificial lung organoid.

54. The method of claim 53, wherein the response comprises fibrosis formation; cell death, cell growth, absorption of the agent, distribution of the agent, metabolism of the agent, excretion of the agent, and/or upregulation or downregulation of a substance by the at least one cell.

55. The artificial lung organoid of any of claims 43-45, wherein the lung organoid differentiates into alveolar like cells after a withdrawal of CHIR99021 and KGF and in presence of SB431542.

56. The artificial lung organoid of any of claims 43-45, wherein the lung organoid differentiates into airway basal like cells when cultured on feeder cells in presence of an inhibitor of Rho kinase (ROCK) and EGF.

57. The method of any of claims 1-29, wherein step (d) further comprises generating a single cell suspension before culturing the dissociated LBO or BLBO in the 3D matrix.
